(19) United States
(12) Patent Application Publication
(10) Pub. No.: US 2017/0174788 A1
(43) Pub. Date: Jun. 22, 2017

(54) COMBINATION OF A JAK INHIBITOR AND AN MMP9 BINDING PROTEIN FOR TREATING INFLAMMATORY DISORDERS

(71) Applicant: Gilead Sciences, Inc., Foster City, CA (US)

(72) Inventors: Sunhwa Kim, San Mateo, CA (US); Victoria Smith, Burlingame, CA (US)

(21) Appl. No.: 15/380,916

(22) Filed: Dec. 15, 2016

Related U.S. Application Data
(60) Provisional application No. 62/366,407, filed on Jul. 25, 2016.
(60) Provisional application No. 62/269,072, filed on Dec. 17, 2015.

(51) Int. Cl. C07K 16/40; A61K 31/541; A61K 39/395

(52) U.S. Cl. CPC C07K 16/40; C12Y 304/24035; A61K 39/3955; A61K 31/541; C07K 2317/565; C07K 2317/76; C07K 2317/56; C07K 2317/34; A61K 2039/505

(57) ABSTRACT

Provided herein are methods and pharmaceutical compositions for the treatment of inflammatory disorders comprising filgotinib and a matrix metalloproteinase-9 (MMP9) binding protein.

### CROSS REFERENCE TO RELATED APPLICATIONS

This application claims the benefit under 35 U.S.C. §119(e) of U.S. Provisional Application No. 62/269,072, filed Dec. 17, 2015, and U.S. Provisional Application No. 62/366,407, filed Jul. 25, 2016, the entireties of which are incorporated herein by reference.

### BACKGROUND

Janus kinase (JAK) is a family of intracellular, nonreceptor tyrosine kinases that transduce cytokine-mediated signals via the JAK-STAT pathway. Filgotinib is a JAK1 selective inhibitor.

The matrix metalloproteinase (MMP) family of extracellular enzymes is involved in forming and remodeling the extracellular matrix. These enzymes contain a conserved catalytic domain in which a zinc atom is coordinated by three histidine residues. Over 20 members of this family are known, organized into a number of groups including collagenases, gelatinases, stromelysins, matrilysins, enamelysins and membrane MMPs. MMP9 belongs to the gelatinase group of MMPs. The gelatinases contain signal peptide, propeptide, catalytic, zinc-binding and heamopexin-like domains common to most MMPs, as well as a plurality of fibronectin-like domains and an O-glycosylated domain.

### SUMMARY

Disclosed herein are combination therapies for the treatment of inflammatory disorders comprising administration of filgotinib or a pharmaceutically acceptable salt, solvate, polymorph, or metabolite thereof, and an MMP9 binding protein (e.g., an anti-MMP9 antibody or fragment thereof). In some embodiments, the inflammatory disorder is selected from rheumatoid arthritis, Crohn's disease, and ulcerative colitis.

### BRIEF DESCRIPTION OF THE DRAWINGS

FIG. 1 shows the amino acid sequence of the heavy chain variable region of a mouse monoclonal anti-MMP9 antibody (AB0041), along with the amino acid sequences of humanized variants of heavy chain (VH1-VH4), aligned to show differences in framework amino acid sequence resulting from humanization. CDRs are shown in italics, and amino acids that are different in the humanized variants, compared to the parent mouse monoclonal, are underlined.

FIG. 2 shows the amino acid sequence of the light chain variable region of a mouse monoclonal anti-MMP9 antibody (AB0041), along with the amino acid sequences of humanized variants of this light chain (Vk1-Vk4), aligned to show differences in framework amino acid sequence resulting from humanization. CDRs are shown in italics, and amino acids that are different in the humanized variants, compared to the parent mouse monoclonal, are underlined.

FIG. 3 shows a schematic diagram of the MMP9 protein.

FIG. 4 shows a comparison between the amino acid sequences of the heavy and light chains of antibodies designated AB0041, M4, and M12.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

Practice of the present disclosure employs, unless otherwise indicated, standard methods and conventional techniques in the fields of cell biology, toxicology, molecular biology, biochemistry, cell culture, immunology, oncology, recombinant DNA and related fields as are within the skill of the art. Such techniques are described in the literature and thereby available to those of skill in the art. See, for example, Alberts, B. et al., “Molecular Biology of the Cell,” 5^{th} edition, Garland Science, New York, N.Y., 2008; Voet, D. et al. “Fundamentals of Biochemistry: Life at the Molecular Level,” 3^{rd} edition, John Wiley & Sons, Hoboken, N.J., 2008; Sambrook, J. et al., “Molecular Cloning: A Laboratory Manual,” 3^{rd} edition, Cold Spring Harbor Laboratory Press, 2001; Ausubel, F. et al., “Current Protocols in Molecular Biology,” John Wiley & Sons, New York, 1987 and periodic updates; Freshney, R.I., “Culture of Animal Cells: A Manual of Basic Technique,” 4^{th} edition, John Wiley & Sons, Somerset, N J, 2000; and the series “Methods in Enzymology,” Academic Press, San Diego, Calif. See also, for example, “Current Protocols in Immunology,” (R. Coico, series editor), Wiley, last updated August 2010.

### DEFINITIONS

As used in the present specification, the following words, phrases and symbols are generally intended to have the meanings as set forth below, except to the extent that the context in which they are used indicates otherwise.

Reference to the word “comprise” and variations thereof, such as, “comprises” and “comprising” are to be construed in an open, inclusive sense, that is, as “including, but not limited to.” Further, the singular forms “a,” “an,” and “the” include plural references unless the context clearly dictates otherwise. Thus, reference to “the compound” includes a plurality of such compounds, and reference to “the assay” includes reference to one or more assays and equivalents thereof known to those skilled in the art.

Reference to “about” a value or parameter herein includes (and describes) embodiments that are directed to that value or parameter per se. The term “about X” thus includes description of “X”. In certain embodiments, the term “about” includes the indicated amount ±10%. In other embodiments, the term “about” includes the indicated amount ±5%. In certain other embodiments, the term “about” includes the indicated amount ±1%.

Recitation of numeric ranges of values throughout the specification is intended to serve as a shorthand notation of referring individually to each separate value falling within the range inclusive of the values defining the range, and each separate value is incorporated in the specification as it were individually recited herein.

As used herein, the term “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable vehicles (e.g., carriers, adjuvants, and/or other excipients) have preferably met the required standards of toxicological and manufacturing testing and/or are included on the Inactive Ingredient Guide prepared by the U.S. Food and Drug administration.

“Pharmaceutically acceptable salts” include, for example, salts with inorganic acids and salts with an organic acid. Examples of salts may include hydrochlorate, phosphate, diphosphate, hydrobromate, sulfate, sulfinate, nitrate, malate, maleate, fumarate, tartrate, succinate, citrate, acetate, lactate, mesylate, p-toluenesulfonate, 2-hydroxyethylsulfonate, benzoate, salicylate, stearate, and alkanoate (such as acetate, HOOC—(CH2)n-COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The term “co-crystal” refers to a crystalline material formed by combining a compound, such as those disclosed herein, and one or more co-crystal formers (i.e., a molecule, ion or atom). In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are known to those of skill in the art.

The term “polymorph” refers to different crystal structures of a crystalline compound. The different polymorphs may result from differences in crystal packing (packing polymorphism) or differences in packing between different conformers of the same molecule (conformational polymorphism).

The term “solvate” refers to an association or complex of one or more solvent molecules and a compound of the disclosure. Examples of solvents that form solvates may include water, isopropanol, ethanol, methanol, dimethylsulfoxide, ethylacetate, acetic acid and ethanolamine.

The term “hydrate” refers to the complex formed by the combining of a compound described herein and water.

The term “prodrug” is defined in the pharmaceutical field as a biologically inactive derivative of a drug that upon administration to the human body is converted to the biologically active parent drug according to some chemical or enzymatic pathway.

The term “racemates” refers to a mixture of enantiomers.

The terms “stereoisomer” or “stereoisomers” refer to compounds that differ in the chirality of one or more stereocenters. Stereoisomers include enantiomers and diastereomers. The compounds may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see, e.g., Chapter 4 of Advanced Organic Chemistry, 4th ed., J. March, John Wiley and Sons, New York, 1992).

For any amino acid and nucleotide sequence disclosed here, its biological variants are also disclosed. A biological variant can be a sequence that has certain level of sequence identity to a reference sequence disclosed. For instance, a biological variant of a protein sequence can be an amino sequence having at least about 70%, 75%, 80%, 85%, 90%, 95%, 96%, 97%, 98%, or 99% sequence identity to the protein. In some embodiments, the amino acid sequence retains one or more desired biological, structural or sequential characteristic of the protein. In another embodiment, a biological variant can be obtained with one, two or three addition, deletion or substitution from the reference protein.

“Homology” or “identity” or “similarity” as used herein in the context of nucleic acids and polypeptides refers to the relationship between two polypeptides or two nucleic acid molecules based on an alignment of the amino acid sequences or nucleic acid sequences, respectively. Homology and identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. In comparing two sequences, the absence of residues (amino acids or nucleic acids) or presence of extra residues also decreases the identity and homology/similarity.

As used herein, “identity” means the percentage of identical nucleotide or amino acid residues at corresponding positions in two or more sequences when the sequences are aligned to maximize sequence matching, i.e., taking into account gaps and insertions. Sequences are generally aligned for maximum correspondence over a designated region, e.g., a region at least about 20, 25, 30, 35, 40, 45, 50, 55, 60, 65 or more amino acids or nucleotides in length, and can be up to the full-length of the reference amino acid or nucleotide. For sequence comparison, typically one sequence acts as a reference sequence, to which test sequences are compared. When using a sequence comparison algorithm, test and reference sequences are input into a computer program, subsequence coordinates are designated, if necessary, and sequence algorithm program parameters are designated. The sequence comparison algorithm then calculates the percent sequence identity for the test sequence(s) relative to the reference sequence, based on the designated program parameters.

Examples of algorithms that are suitable for determining percent sequence identity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (www.ncbi.nlm.nih.gov). Further exemplary algorithms include ClustalW (Higgins D., et al. (1994) Nucleic Acids Res 22: 4673-4680), available at www.ebi.ac.uk/Tools/clustalw/index.html.

Residue positions which are not identical can differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine.

Sequence identity between two nucleic acids can also be described in terms of hybridization of two molecules to each other under stringent conditions. The hybridization conditions are selected following standard methods in the art (see, for example, Sambrook, et al., Molecular Cloning: A Laboratory Manual, Second Edition, (1989) Cold Spring Harbor, N.Y.). An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 mg/ml denatured, sheared salmon sperm DNA, followed by washing the filters in 0.1×SSC at about 65° C. Stringent hybridization conditions are hybridization conditions that are at least as stringent as the above representative conditions, where conditions are considered to be at least as stringent if they are at least about 80% as stringent, typically at least 90% as stringent as the above specific stringent conditions.

### Combination Treatments

The present disclosure, in some embodiments, provides compositions, formulations, medicaments, use and kits for inflammatory disorders. The treatments may entail the administration to a patient filgotinib, or a pharmaceutically acceptable salt, solvate, polymorph, or metabolite thereof and an MMP9 binding protein, e.g., an anti-matrix metalloproteinase 9 (MMP9) antibody including a selective anti-MMP9 antibody.

Filgotinib, or a pharmaceutically acceptable salt, solvate, polymorph, or metabolite thereof, and an MMP9 binding protein may be administered separately or concurrently to a subject. In some embodiments, filgotinib, or a pharmaceutically acceptable salt, solvate, polymorph, or metabolite thereof, and an MMP9 binding protein are conjugated to form an antibody-drug conjugate (ADC).

The treatment may be administered to a mammal subject, in particular a human subject. Inflammatory disorders that may be suitably treated include, without limitation, inflammatory bowel disease (IBD) (including Crohn's disease, ulcerative colitis (UC), and indeterminate colitis), collagenous colitis, rheumatoid arthritis, osteoarthritis, septicemia, sepsis, psoriasis, myestenia gravis, acute disseminated encephalomyelitis, idiopathic thrombocytopenic purpura, Sjoegren's syndrome, autoimmune hemolytic anemia, multiple sclerosis, muscular dystrophy, lupus, allergy, asthma, chronic obstructive pulmonary disease (COPD), non-alcoholic steatohepatitis (NASH), and metabolic disorders characterized by impaired insulin production and glucose intolerance (e.g., Insulin Dependent Diabetes Mellitus (IDDM, also known as type 1 diabetes), and Non-Insulin-Dependent Diabetes Mellitus (NIDDM, also known as type 2 diabetes)).

### Filgotinib

Filgotinib, also known as GLPG0634, is described in U.S. Pat. No. 8,563,545, and has the following structure:

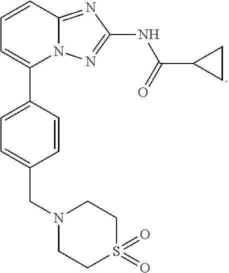 [SMILES: O=C(Nc1nc2cccc(-c3ccc(CN4CCS(=O)(=O)CC4)cc3)n2n1)C1CC1]

Solvates and polymorphs of filgotinib are described in International Application Publication No. WO 2015/117981. A metabolite of filgotinib is described in U.S. Pat. No. 9,284,311. One specific pharmaceutically acceptable salt of filgotinib that may be used in the methods and compositions disclosed herein is the maleate salt of filgotinib.

### MMP9 Binding Proteins

Abnormal activity of certain MMPs plays a role in tumor growth, metastasis, inflammation, autoimmunity, and vascular disease. One of the MMPs, matrix metalloproteinase-9 (MMP9), also known as gelatinase-B, degrades basement membrane collagen and other extracellular matrix (ECM) components.

An anti-MMP antibody is an antibody that recognizes an MMP (an “anti-MMP antibody”). An anti-MMP antibody may be a pan anti-MMP antibody which recognizes all or most members of the MMP family of proteins, a non-selective anti-MMP antibody that recognizes one or more MMP family members, or a selective anti-MMP antibody such as a selective anti-MMP9 antibody.

A selective anti-MMP9 antibody binds preferentially to MMP9 relative to other MMPs such as MMP1, MMP2, MMP3, MMP7, MMP9, MMP10, MMP12, and MMP13. A selective anti-MMP9 antibody is generally not significantly or detectably cross-reactive with other MMPs. A selective anti-MMP9 antibody may not affect the activity of other MMPs. A selective anti-MMP9 antibody, in some embodiments, inhibits the enzymatic processing or inhibiting action of MMP9 on it substrate (e.g., by inhibiting substrate binding, substrate cleavage, and the like).

An anti-MMP9 antibody may specifically recognize a mouse MMP9 or a non-mouse MMP9, such as human MMP9, Cynomolgus monkey MMP9, and rat MMP9.

An anti-MMP9 antibody may be a non-competitive inhibitor to MMP9. A “non-competitive inhibitor” refers to an inhibitor that binds at a site away from the substrate binding site of an enzyme, and thus can bind the enzyme and effect inhibitory activity regardless of whether or not the enzyme is bound to its substrate. Such non-competitive inhibitors can, for example, provide for a level of inhibition that can be substantially independent of substrate concentration.

In some embodiments, a selective anti-MMP9 antibody specifically binds to, and inhibits the catalytic activity of, MMP9. The binding of a specific selective anti-MMP9 antibody may lead to modulation (e.g., inhibition) of MMP9, e.g., without directly or substantially affecting the activity of other MMPs.

As used herein, the term “antibody” means an isolated or recombinant polypeptide binding agent that comprises peptide sequences (e.g., variable region sequences) that specifically bind an antigenic epitope. The term is used in its broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including but not limited to Fv, scFv, Fab, Fab′ F(ab′)₂ and Fab₂, so long as they exhibit the desired biological activity. The term “human antibody” refers to antibodies containing sequences of human origin, except for possible non-human CDR regions, and does not imply that the full structure of an immunoglobulin molecule be present, only that the antibody has minimal immunogenic effect in a human (i.e., does not induce the production of antibodies to itself).

An “antibody fragment” comprises a portion of a full-length antibody, for example, the antigen binding or variable region of a full-length antibody. Such antibody fragments may also be referred to herein as “functional fragments: or “antigen-binding fragments.” Examples of antibody fragments include Fab, Fab′, F(ab′)₂, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is a minimum antibody fragment containing a complete antigen-recognition and -binding site. This region consists of a dimer of one heavy- and one light-chain variable domain in tight, non-covalent association. It is in this configuration that the three complementarity-determining regions (CDRs) of each variable domain interact to define an antigen-binding site on the surface of the V_{H}-V_{L} dimer. Collectively, the six CDRs confer antigen-binding specificity to the antibody. However, even a single variable domain (or an isolated V_{H} or V_{L} region comprising only three of the six CDRs specific for an antigen) has the ability to recognize and bind antigen, although generally at a lower affinity than does the entire Fᵥ fragment.

The “F_{ab}” fragment also contains, in addition to heavy and light chain variable regions, the constant domain of the light chain and the first constant domain (CHO of the heavy chain. Fab fragments were originally observed following papain digestion of an antibody. Fab′ fragments differ from Fab fragments in that F(ab′) fragments contain several additional residues at the carboxy terminus of the heavy chain CH₁ domain, including one or more cysteines from the antibody hinge region. F(ab′)₂ fragments contain two Fab fragments joined, near the hinge region, by disulfide bonds, and were originally observed following pepsin digestion of an antibody. Fab′-SH is the designation herein for Fab′ fragments in which the cysteine residue(s) of the constant domains bear a free thiol group. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to five major classes: IgA, IgD, IgE, IgG, and IgM, and several of these may be further divided into subclasses (isotypes), e.g., IgG1, IgG2, IgG3, IgG4, IgA1, and IgA2.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun, in The Pharmacology of Monoclonal Antibodies, vol. 113 (Rosenburg and Moore eds.) Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain, thereby creating two antigen-binding sites. Diabodies are additionally described, for example, in EP 404,097; WO 93/11161 and Hollinger et al. (1993) Proc. Natl. Acad. Sci. USA 90:6444-6448.

An “isolated” antibody or fragment thereof is one that has been identified and separated and/or recovered from a component of its natural environment. Components of its natural environment may include enzymes, hormones, and other proteinaceous or nonproteinaceous solutes. In some embodiments, an isolated antibody or fragment thereof is purified (1) to greater than 95% by weight of antibody or fragment thereof as determined by the Lowry method, for example, more than 99% by weight, (2) to a degree sufficient to obtain at least 15 residues of N-terminal or internal amino acid sequence, e.g., by use of a spinning cup sequenator, or (3) to homogeneity by gel electrophoresis (e.g., SDS-PAGE) under reducing or nonreducing conditions, with detection by Coomassie blue or silver stain. The term “isolated antibody” or fragment thereof includes an antibody or fragment thereof in situ within recombinant cells, since at least one component of the antibody's natural environment will not be present. In certain embodiments, an isolated antibody or fragment thereof is prepared by at least one purification step.

As used herein, “immunoreactive” refers to antibodies or fragments thereof that are specific to a sequence of amino acid residues (“binding site” or “epitope”), yet if are cross-reactive to other peptides/proteins, are not toxic at the levels at which they are formulated for administration to human use. “Epitope” refers to that portion of an antigen capable of forming a binding interaction with an antibody or fragment thereof. An epitope can be a linear peptide sequence (i.e., “continuous”) or can be composed of noncontiguous amino acid sequences (i.e., “conformational” or “discontinuous”). The term “preferentially binds” means that the binding agent binds to the binding site with greater affinity than it binds unrelated amino acid sequences.

Anti-MMP9 antibodies or fragments thereof can be described in terms of the CDRs of the heavy and light chains. As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest’ (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison.

TABLE 1
CDR Definitions:

| | Kabat¹ | Chothia² | MacCallum³ |
|---|---|---|---|
| V_{H} CDR1 | 31-35 | 26-32 | 30-35 |
| V_{H} CDR2 | 50-65 | 53-55 | 47-58 |
| V_{H} CDR3 | 95-102 | 96-101 | 93-101 |
| V_{L} CDR1 | 24-34 | 26-32 | 30-36 |
| V_{L} CDR2 | 50-56 | 50-52 | 46-55 |
| V_{L} CDR3 | 89-97 | 91-96 | 89-96 |

¹Residue numbering follows the nomenclature of Kabat et al., supra ²Residue numbering follows the nomenclature of Chothia et al., supra ³Residue numbering follows the nomenclature of MacCallum et al., supra

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

In some embodiments, an antibody or fragment thereof, as disclosed herein, is a humanized antibody or fragment thereof, or a human antibody or fragment thereof. Humanized antibodies or fragments thereof include human immununoglobulins (recipient antibody) in which residues from a complementary-determining region (CDR) of the recipient are replaced by residues from a CDR of a non-human species (donor antibody) such as mouse, rat or rabbit having the desired specificity, affinity and capacity. Thus, humanized forms of non-human (e.g., murine) antibodies or fragments thereof are chimeric immunoglobulins which contain minimal sequence derived from non-human immunoglobulin. The non-human sequences are located primarily in the variable regions, particularly in the complementarity-determining regions (CDRs). In some embodiments, Fv framework residues of the human immunoglobulin are replaced by corresponding non-human residues. Humanized antibodies or fragments thereof can also comprise residues that are found neither in the recipient antibody nor in the imported CDR or framework sequences. In certain embodiments, a humanized antibody or fragment thereof comprises substantially all of at least one, and typically two, variable domains, in which all or substantially all of the CDRs correspond to those of a non-human immunoglobulin and all or substantially all of the framework regions are those of a human immunoglobulin consensus sequence. For the purposes of the present disclosure, humanized antibodies or fragments thereof can also include immunoglobulin fragments, such as Fv, Fab, Fab′, F(ab′)₂ or other antigen-binding subsequences of antibodies.

The humanized antibody or fragment thereof can also comprise at least a portion of an immunoglobulin constant region (Fc), typically that of a human immunoglobulin. See, for example, Jones et al. (1986) Nature 321:522-525; Riechmann et al. (1988) Nature 332:323-329; and Presta (1992) Curr. Op. Struct. Biol. 2:593-596.

Methods for humanizing non-human antibodies or fragments thereof are known in the art. Generally, a humanized antibody or fragment thereof has one or more amino acid residues introduced into it from a source that is non-human. These non-human amino acid residues are often referred to as “import” or “donor” residues, which are typically obtained from an “import” or “donor” variable domain. For example, humanization can be performed essentially according to the method of Winter and co-workers, by substituting rodent CDRs or CDR sequences for the corresponding sequences of a human antibody. See, for example, Jones et al., supra; Riechmann et al., supra and Verhoeyen et al. (1988) Science 239:1534-1536. Accordingly, such “humanized” antibodies or fragments thereof include chimeric antibodies (U.S. Pat. No. 4,816,567), wherein substantially less than an intact human variable domain has been substituted by the corresponding sequence from a non-human species. In certain embodiments, humanized antibodies or fragments thereof are human antibodies in which some CDR residues and optionally some framework region residues are substituted by residues from analogous sites in rodent antibodies (e.g., murine monoclonal antibodies).

Human antibodies or fragments thereof can also be produced, for example, by using phage display libraries. Hoogenboom et al. (1991) J. Mol. Biol, 227:381; Marks et al. (1991) J. Mol. Biol. 222:581. Other methods for preparing human monoclonal antibodies are described by Cole et al. (1985) “Monoclonal Antibodies and Cancer Therapy,” Alan R. Liss, p. 77 and Boerner et al. (1991) J. Immunol. 147:86-95.

Human antibodies or fragments thereof can be made by introducing human immunoglobulin loci into transgenic animals (e.g., mice) in which the endogenous immunoglobulin genes have been partially or completely inactivated. Upon immunological challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al. (1992) Bio/Technology 10:779-783 (1992); Lonberg et al. (1994) Nature 368: 856-859; Morrison (1994) Nature 368:812-813; Fishwald et al. (1996) Nature Biotechnology 14:845-851; Neuberger (1996) Nature Biotechnology 14:826; and Lonberg et al. (1995) Intern. Rev. Immunol. 13:65-93.

An antibody or fragment thereof, as disclosed herein, can be affinity matured using known selection and/or mutagenesis methods as described above. In some embodiments, affinity matured antibodies or fragments thereof have an affinity which is five times or more, ten times or more, twenty times or more, or thirty times or more than that of the starting antibody or fragment thereof (generally murine, rabbit, chicken, humanized or human) from which the matured antibody or fragment thereof is prepared.

An antibody or fragment thereof, as disclosed herein, can also be a bispecific antibody. Bispecific antibodies or fragments thereof are monoclonal, and may be human or humanized antibodies or fragments thereof that have binding specificities for at least two different antigens. In the present case, the two different binding specificities can be directed to two different MMPs, or to two different epitopes on a single MMP (e.g., MMP9).

An antibody or fragment thereof, as disclosed herein, can also be an immunoconjugate. Such immunoconjugates comprise an antibody or fragment thereof (e.g., to MMP9) conjugated to a second molecule, such as a reporter. An immunoconjugate can also comprise an antibody or fragment thereof conjugated to a cytotoxic agent such as a chemotherapeutic agent, a toxin (e.g., an enzymatically active toxin of bacterial, fungal, plant, or animal origin, or fragments thereof), or a radioactive isotope (i.e., a radioconjugate).

An antibody or fragment thereof that “specifically binds to” or is “specific for” a particular polypeptide or an epitope refers to the selective binding of the antibody or fragment thereof to the target antigen or epitope; these terms, and methods for determining specific binding, are well understood in the art. An antibody or fragment thereof exhibits “specific binding” for a particular target antigen or epitope if it binds with greater affinity, avidity, more readily, and/or with greater duration to that target antigen or epitope than it does with other substances. In some embodiments, the antibody or fragment thereof that specifically binds to the polypeptide or epitope is one that that binds to that particular polypeptide or epitope without substantially binding to any other polypeptide or polypeptide epitope. In some embodiments, the antibodies or fragments thereof, as disclosed herein, specifically bind to human MMP9 with a dissociation constant (K_{d}) equal to or lower than 100 nM, optionally lower than 10 nM, optionally lower than 1 nM, optionally lower than 0.5 nM, optionally lower than 0.1 nM, optionally lower than 0.01 nM, or optionally lower than 0.005 nM, in certain examples, between 0.1 and 0.2 nM, or between 0.1 and 10 pM, e.g., between 0.4 and 9 pm, such as between 0.4 and 8.8 pm, in the form of monoclonal antibody, scFv, Fab, or other form of antibody measured at a temperature of about 4° C., 25° C., 37° C. or 42° C.

In certain embodiments, the antibodies or fragments thereof, as disclosed herein, bind to one or more processing sites (e.g., sites of proteolytic cleavage) in MMP9, thereby effectively blocking processing of the proenzyme or preproenzyme to the catalytically active enzyme, and thus reducing the proteolytic activity of the MMP9.

In certain embodiments, the antibodies or fragments thereof, as disclosed herein bind to MMP9 with an affinity at least 2 times, at least 5 times, at least 10 times, at least 25 times, at least 50 times, at least 100 times, at least 500 times, or at least 1000 times greater than its binding affinity for another MMP. Binding affinity can be measured by any method known in the art and can be expressed as, for example, on-rate, off-rate, dissociation constant (K_{d}), equilibrium constant (K_{eq}) or any term in the art.

In certain embodiments, the antibodies or fragments thereof, as disclosed herein, inhibit the enzymatic (i.e., catalytic) activity of MMP9 such as by non-competitive inhibition. In certain embodiments, the antibodies or fragments thereof, as disclosed herein, bind within the catalytic domain of MMP9. In additional embodiments, the antibodies or fragments thereof, as disclosed herein, bind outside the catalytic domain of MMP9.

In some embodiments, the antibodies or fragments disclosed herein comprise: a heavy chain variable (VH) region comprising a heavy chain complementary determining region (CDR) with an amino acid sequence selected from the group consisting of SEQ ID NO: 13, SEQ ID NO: 14, SEQ ID NO: 15, and combinations thereof. In one embodiment, the VH region comprises a heavy chain CDR1 with the amino acid sequence of SEQ ID NO: 13, a heavy chain CDR2 with the amino acid sequence of SEQ ID NO: 14, and a heavy chain CDR3 with the amino acid sequence of SEQ ID NO: 15.

In some embodiments, the antibodies or fragments disclosed herein comprise: a VH region comprising a CDR with an amino acid sequence selected from the group consisting of: SEQ ID NO: 34, SEQ ID NO: 35, SEQ ID NO: 36, and combinations thereof. In one embodiment, the VH region comprises a heavy chain CDR1 with the amino acid sequence of SEQ ID NO: 34, a heavy chain CDR2 with the amino acid sequence of SEQ ID NO: 35, and a heavy chain CDR3 with the amino acid sequence of SEQ ID NO: 36.

In some embodiments, the antibodies or fragments disclosed herein comprise: a VH region comprising the amino acid sequence set forth in SEQ ID NO: 1, SEQ ID NO: 3, SEQ ID NO: 5, SEQ ID NO: 6, SEQ ID NO: 7, SEQ ID NO: 8, SEQ ID NO: 32, or SEQ ID NO: 47.

In some embodiments, the antibodies or fragments disclosed herein comprise: a light chain variable (VL) region comprising a CDR with an amino acid sequence selected from the group consisting of: SEQ ID NO: 16, SEQ ID NO: 17, SEQ ID NO: 18, and combinations thereof. In one embodiment, the VL region comprises a light chain CDR1 with the amino acid sequence of SEQ ID NO: 16, a light chain CDR2 with the amino acid sequence of SEQ ID NO: 17, and a light chain CDR3 with the amino acid sequence of SEQ ID NO: 18.

In some embodiments, the antibodies or fragments disclosed herein comprise: a VL region comprising a CDR with an amino acid sequence selected from the group consisting of: SEQ ID NO: 37, SEQ ID NO: 38, SEQ ID NO: 39, and combinations thereof. In one embodiment, the VL region comprises a light chain CDR1 with the amino acid sequence of SEQ ID NO: 37, a light chain CDR2 with the amino acid sequence of SEQ ID NO: 38, and a light chain CDR3 with the amino acid sequence of SEQ ID NO: 39.

In some embodiments, the antibodies or fragments disclosed herein comprise: a VL region comprising a CDR with an amino acid sequence selected from the group consisting of: SEQ ID NO: 42, SEQ ID NO: 43, SEQ ID NO: 44, and combinations thereof. In one embodiment, the VL region of the isolated antibody or fragment thereof, as disclosed herein, comprises a light chain CDR1 with the amino acid sequence of SEQ ID NO: 42, a light chain CDR2 with the amino acid sequence of SEQ ID NO: 43, and a light chain CDR3 with the amino acid sequence of SEQ ID NO: 44.

In some embodiments, the antibodies or fragments disclosed herein comprise: a VL region comprising the amino acid sequence set forth in SEQ ID NO: 2, SEQ ID NO: 4, SEQ ID NO: 9, SEQ ID NO: 10, SEQ ID NO: 11, SEQ ID NO: 12, SEQ ID NO: 33, SEQ ID NO: 41, or SEQ ID NO: 48.

In another embodiment, the antibodies or fragments disclosed herein comprise: a VH region of SEQ ID NO: 7 or at least about 75%, 80%, 85%, 90%, 91%, 92%, 93%, 94%, 95%, 96%, 97%, 98%, 99% or more sequence identity with SEQ ID NO: 7; and/or a VL region of SEQ ID NO: 12 or at least at or about 75%, 80%, 85%, 90%, 91%, 92%, 93%, 94%, 95%, 96%, 97%, 98%, 99% or more sequence identity with SEQ ID NO: 12.

In another embodiment, the antibodies or fragments disclosed herein comprise: a VH region with an amino acid sequence set forth in SEQ ID NO: 32 or SEQ ID NO: 47 or at least about 75%, 80%, 85%, 90%, 91%, 92%, 93%, 94%, 95%, 96%, 97%, 98%, 99% or more sequence identity with SEQ ID NO: 32 or SEQ ID NO: 47; and/or a VL region with an amino acid sequence set forth in SEQ ID NO: 33, SEQ ID NO: 41 or in SEQ ID NO: 48 or at least about 75%, 80%, 85%, 90%, 91%, 92%, 93%, 94%, 95%, 96%, 97%, 98%, 99% or more sequence identity with SEQ ID NO: 33, SEQ ID NO: 41 or SEQ ID NO: 48.

In some embodiments, the antibodies or fragments disclosed herein comprise: a VH region with an amino acid sequence set forth in SEQ ID NO: 1 or at least at about 75%, 80%, 85%, 90%, 91%, 92%, 93%, 94%, 95%, 96%, 97%, 98%, 99% or more sequence identity with SEQ ID NO: 1; and/or a VL region with an amino acid sequence set forth in SEQ ID NO: 2, or at least about 75%, 80%, 85%, 90%, 91%, 92%, 93%, 94%, 95%, 96%, 97%, 98%, 99% or more sequence identity with SEQ ID NO: 2.

Also provided in the present disclosure are antibodies or fragments thereof that compete with any of the anti-MMP9 antibodies or antigen binding fragments thereof described herein for binding to MMP9. For instance, in some embodiments, the anti-MMP9 antibodies or functional fragments thereof, as disclosed herein, compete for binding with an antibody having a heavy chain polypeptide of any of SEQ ID NOS: 1 or 5-8, a light chain polypeptide of SEQ ID NOS: 2 or 9-12, or combinations thereof. In one embodiment, the antibodies or fragments thereof compete for binding to MMP9 with an antibody comprising a VH region with the amino acid sequence set forth in SEQ ID. NO: 7, and/or a VL with the amino acid sequence as set forth in SEQ ID. NO: 12. In another embodiment, the anti-MMP9 antibodies or fragments thereof, as disclosed herein, compete for binding to human MMP9 with the antibody described herein as AB0041.

Also provided in the present disclosure are antibodies and fragments thereof that bind to the same epitope, e.g., MMP9 epitope, as any one or more of the antibodies described herein. Accordingly, the present disclosure provides, in some embodiments, antibodies or fragments thereof that specifically bind to an epitope of MMP9, where the epitope comprises an amino acid residue within a particular region of MMP9 or multiple regions of MMP9. Such regions can include, for example, structural loops and/or other structural domains of MMP9, such as those shown to be important for binding to exemplary antibodies described herein. Typically, the regions are defined according to amino acid residue positions on the full-length MMP9 sequence, e.g., SEQ ID NO: 27. In one embodiment, the epitope comprises an amino acid (i.e., one or more amino acid residue(s)) residue 104-202 of SEQ ID NO: 27. In another example, the epitope comprises an amino acid residue within a region that is residues 104-119 residues 159-166, or residues 191-202 of SEQ ID NO: 27. In yet another embodiment, the epitope comprises an amino acid within a region of MMP9 that is residues 104-119 of SEQ ID NO: 27, an amino acid residue within a region of MMP9 that is residues 159-166 of SEQ ID NO: 27, and an amino acid residue within a region of MMP9 that is residues 191-202 of SEQ ID NO: 27. In additional embodiments, the epitope comprises E111, D113, R162, or 1198 of SEQ ID NO: 27. In one such embodiment, the epitope comprises R162 of SEQ ID NO: 27.

### MMP9 Sequence

The amino acid sequence of the human MMP9 protein is as follows:

| (SEQ ID NO: 27) | |
|---|---|
| MSLWQPLVLV LLVLGCCFAA PRQRQSTLVL FPGDLRTNLT DRQLAEEYLY | 50 |
| RYGYTRVAEM RGESKSLGPA LLLLQKQLSL PETGELDSAT LKAMRTPRCG | 100 |
| VPDLGRFQTF EGDLKWHHHN ITYWIQNYSE DLPRAVIDDA FARAFALWSA | 150 |
| VTPLTFTRVY SRDADIVIQF GVAEHGDGYP FDGKDGLLAH AFPPGPGIQG | 200 |
| DAHFDDDELW SLGKGVVVPT RFGNADGAAC HFPFIFEGRS YSACTTDGRS | 250 |
| DGLPWCSTTA NYDTDDRFGF CPSERLYTRD GNADGKPCQF PFIFQGQSYS | 300 |
| ACTTDGRSDG YRWCATTANY DRDKLFGFCP TRADSTVMGG NSAGELCVFP | 350 |
| FTFLGKEYST CTSEGRGDGR LWCATTSNFD SDKKWGFCPD QGYSLFLVAA | 400 |
| HEFGHALGLD HSSVPEALMY PMYRFTEGPP LHKDDVNGIR HLYGPRPEPE | 450 |
| PRPPTTTTPQ PTAPPTVCPT GPPTVHPSER PTAGPTGPPS AGPTGPPTAG | 500 |
| PSTATTVPLS PVDDACNVNI FDAIAEIGNQ LYLFKDGKYW RFSEGRGSRP | 550 |
| QGPFLIADKW PALPRKLDSV FEEPLSKKLF FFSGRQVWVY TGASVLGPRR | 600 |
| LDKLGLGADV AQVTGALRSG RGKMLLFSGR RLWRFDVKAQ MVDPRSASEV | 650 |
| DRMFPGVPLD THDVFQYREK AYFCQDRFYW RVSSRSELNQ VDQVGYVTYD | 700 |
| ILQCPED. | |

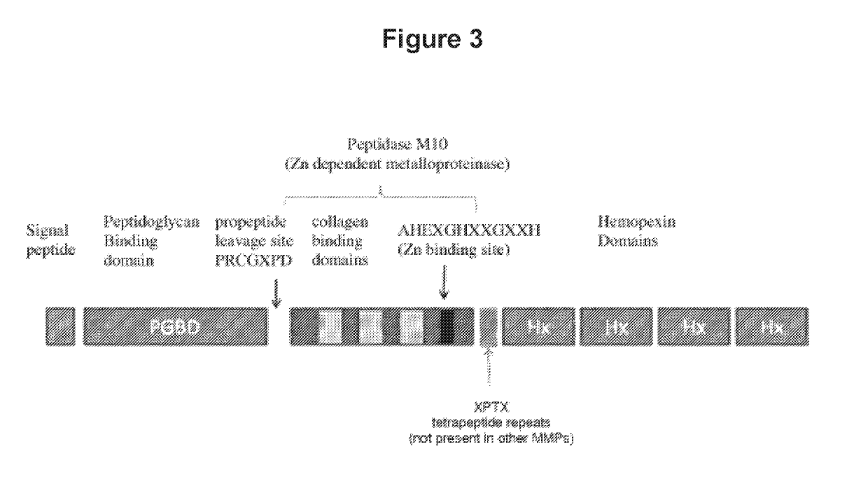

Protein domains are shown schematically in FIG. 3 and are indicated below:

| Amino Acid # | Feature |
|---|---|
| 1-19 | Signal Peptide |
| 38-98 | Peptidoglycan Binding Domain |
| R98/C99 | Cysteine-switch active pocket |
| 112-445 | Zn dependent metalloproteinase domain |
| 223-271 | Fibronectin type II domain (gelatin binding domain) |
| 281-329 | Fibronectin type II domain (gelatin binding domain) |
| 340-388 | Fibronectin type II domain (gelatin binding domain) |
| 400-411 | Zn binding region |
| 521-565 | Hemopexin-like domain |
| 567-608 | Hemopexin-like domain |
| 613-659 | Hemopexin-like domain |
| 661-704 | Hemopexin-like domain. |

The amino acid sequence of mature full-length human MMP9 (which is the amino acid sequence of the propolypeptide of SEQ ID NO: 27 without the signal peptide) is:

(SEQ ID NO: 28)

APRQRQSTLVL FPGDLRTNLT DRQLAEEYLY RYGYTRVAEM
RGESKSLGPA LLLLQKQLSL PETGELDSAT LKAMRTPRCG
VPDLGRFQTF EGDLKWHHHN ITYWIQNYSE DLPRAVIDDA
FARAFALWSA VTPLTFTRVY SRDADIVIQF GVAEHGDGYP
FDGKDGLLAH AFPPGPGIQG DAHFDDDELW SLGKGVVVPT
RFGNADGAAC HFPFIFEGRS YSACTTDGRS DGLPWCSTTA
NYDTDDRFGF CPSERLYTRD GNADGKPCQF PFIFQGQSYS
ACTTDGRSDG YRWCATTANY DRDKLFGFCP TRADSTVMGG
NSAGELCVFP FTFLGKEYST CTSEGRGDGR LWCATTSNFD
SDKKWGFCPD QGYSLFLVAA HEFGHALGLD HSSVPEALMY
PMYRFTEGPP LHKDDVNGIR HLYGPRPEPE PRPPTTTTPQ
PTAPPTVCPT GPPTVHPSER PTAGPTGPPS AGPTGPPTAG
PSTATTVPLS PVDDACNVNI FDAIAEIGNQ LYLFKDGKYW
RFSEGRGSRP QGPFLIADKW PALPRKLDSV FEEPLSKKLF
FFSGRQVWVY TGASVLGPRR LDKLGLGADV AQVTGALRSG
RGKMLLFSGR RLWRFDVKAQ MVDPRSASEV DRMFPGVPLD
THDVFQYREK AYFCQDRFYW RVSSRSELNQ VDQVGYVTYD
ILQCPED.

The amino acid sequence of the signal peptide is MSLWQPLVLVLLVLGCCFA (SEQ ID NO: 29).

Also provided are MMP9 polypeptides, including mutant MMP9 polypeptides. Such peptides are useful, for example, in generating and selecting antibodies and fragments as provided herein. Exemplary polypeptides include those comprising an amino acid sequence comprising residues 111-198 of SEQ ID NO: 27, and those comprising an amino acid sequence comprising residues 111-198 of SEQ ID NO: 27 with an amino acid substitution at residue 111, 113, 162, or 198 of SEQ ID NO 27 or with an amino acid substitution at all such residues. Such polypeptides find use, for example, in selecting antibodies that bind to epitopes comprising such residues and/or for which such residues of MMP9 are important for binding, such as those described herein.

The present disclosure contemplates MMP9 binding proteins that bind any portion of MMP9, e.g., human MMP9, with MMP9 binding proteins that preferentially bind MMP9 relative to other MMPs being of particular interest.

Anti-MMP9 antibodies, and functional fragments thereof, can be generated according to methods well known in the art. Exemplary anti-MMP9 antibodies are provided below.

### Mouse Monoclonal Anti-MMP9 Antibodies

A mouse monoclonal antibody to human MMP9 was obtained. This antibody comprises a mouse IgG2b heavy chain and a mouse kappa light chain, and is denoted AB0041.

The amino acid sequence of the AB0041 heavy chain is as follows:

(SEQ ID NO: 1)

MAVLVLFLCLVAFPSCVLSQVQLKESGPGLVAPSQSLSITCTVSGFSLLS
YGVHWVRQPPGKGLEWLGVIWTGGTTNYNSALMSRLSISKDDSKSQVFLK
MNSLQTDDTAIYYCARYYYGMDYWGQGTSVTVSSAKTTPPSVYPLAPGCG
DTTGSSVTLGCLVKGYFPESVTVTWNSGSLSSSVHTFPALLQSGLYTMSS
SVTVPSSTWPSQTVTCSVAHPASSTTVDKKLEPSGPISTINPCPPCKECH
KCPAPNLEGGPSVFIFPPNIKDVLMISLTPKVTCVVVDVSEDDPDVRISW
FVNNVEVHTAQTQTHREDYNSTIRVVSALPIQHQDWMSGKEFKCKVNNKD
LPSPIERTISKIKGLVRAPQVYILPPPAEQLSRKDVSLTCLVVGFNPGDI
SVEWTSNGHTEENYKDTAPVLDSDGSYFIYSKLDIKTSKWEKTDSFSCNV
RHEGLKNYYLKKTISRSPGK (the signal sequence is
underlined, and the sequence of the IgG2b constant
region is presented italics).

The amino acid sequence of the AB0041 light chain is as follows:

(SEQ ID NO: 2)

MESQIQVFVFVFLWLSGVDGDIVMTQSHKFMSTSVGDRVSITCKASQDVR
NTVAWYQQKTGQSPKLLIYSSSYRNTGVPDRFTGSGSGTDFTFTISSVQA
EDLAVYFCQQHYITPYTFGGGTKLEIKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC (the signal
sequence is underlined, and the sequence of the
kappa constant region is presented in italics).

The following amino acid sequence comprises the framework regions and complementarity-determining regions (CDRs) of the variable region of the IgG2b heavy chain of AB0041 (with CDRs underlined):

(SEQ ID NO: 3)

QVQLKESGPGLVAPSQSLSITCTVSGFSLLSYGVHWVRQPPGKGLEWLGV
IWTGGTTNYNSALMSRLSISKDDSKSQVFLKMNSLQTDDTAIYYCARYYY
GMDYWGQGTSVTVSS.

The following amino acid sequence comprises the framework regions and complementarity-determining regions (CDRs) of the variable region of the kappa light chain of AB0041 (with CDRs underlined):

(SEQ ID NO: 4)

DIVMTQSHKFMSTSVGDRVSITCKASQDVRNTVAWYQQKTGQSPKLLIYS
SSYRNTGVPDRFTGSGSGTDFTFTISSVQAEDLAVYFCQQHYITPYTFGG
GTKLEIK.

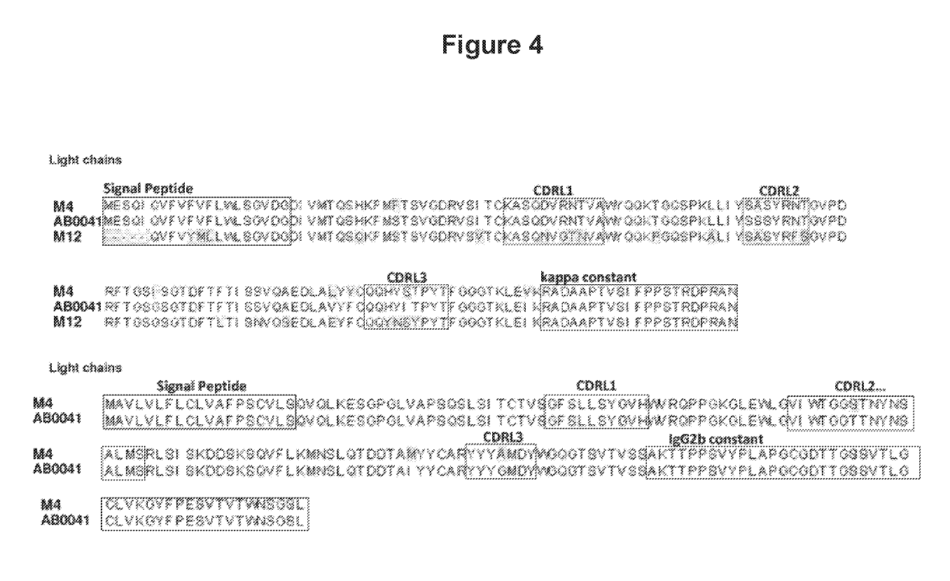

Other exemplary mouse anti-human MMP9 antibodies (e.g., M4 and M12) are described herein. FIG. 4 shows a comparison between the amino acid sequences of the heavy and light chains of antibodies designated AB0041, M4, and M12.

An exemplary anti-mouse MMP9 antibody (AB0046) is also described herein. In some embodiments, provided are uses of such anti-mouse antibodies as surrogate antibodies for testing and assessing the MMP9-inhibition methods, e.g., therapeutic methods, as provided herein.

### Heavy-Chain Variants

The amino acid sequences of the variable regions of the AB0041 heavy and light chains were separately modified, by altering framework region sequences in the heavy and light chain variable regions. The effect of these sequence alterations was to deplete the antibody of human T-cell epitopes, thereby reducing or abolishing its immunogenicity in humans (Antitope, Babraham, UK).

Four heavy-chain variants were constructed, in a human IgG4 heavy chain background containing a S241P amino acid change that stabilizes the hinge domain (Angal et al. (1993) Molec. Immunol. 30:105-108), and are denoted VH1, VH2, VH3 and VH4. The amino acid sequences of their framework regions and CDRs are as follows:

VH1:

(SEQ ID NO: 5)

QVQLQESGPGLVKPSETLSLTCTVSGFSLLSYGVHWVRQPPGKGLEWLGV
IWTGGTTNYNSALMSRLTISKDDSKSTVYLKMNSLKTEDTAIYYCARYYY
GMDYWGQGTSVTVSS;
VH2:

(SEQ ID NO: 6)

QVQLQESGPGLVKPSETLSLTCTVSGFSLLSYGVHWVRQPPGKGLEWLGV
IWTGGTTNYNSALMSRLTISKDDSKNTVYLKMNSLKTEDTAIYYCARYYY
GMDYWGQGTLVTVSS;
VH3:

(SEQ ID NO: 7)

QVQLQESGPGLVKPSETLSLTCTVSGFSLLSYGVHWVRQPPGKGLEWLGV
IWTGGTTNYNSALMSRFTISKDDSKNTVYLKMNSLKTEDTAIYYCARYYY
GMDYWGQGTLVTVSS;
VH4:

(SEQ ID NO: 8)

QVQLQESGPGLVKPSETLSLTCTVSGFSLLSYGVHWVRQPPGKGLEWLGV
IWTGGTTNYNSALMSRFTISKDDSKNTLYLKMNSLKTEDTAIYYCARYYY
GMDYWGQGTLVTVSS.

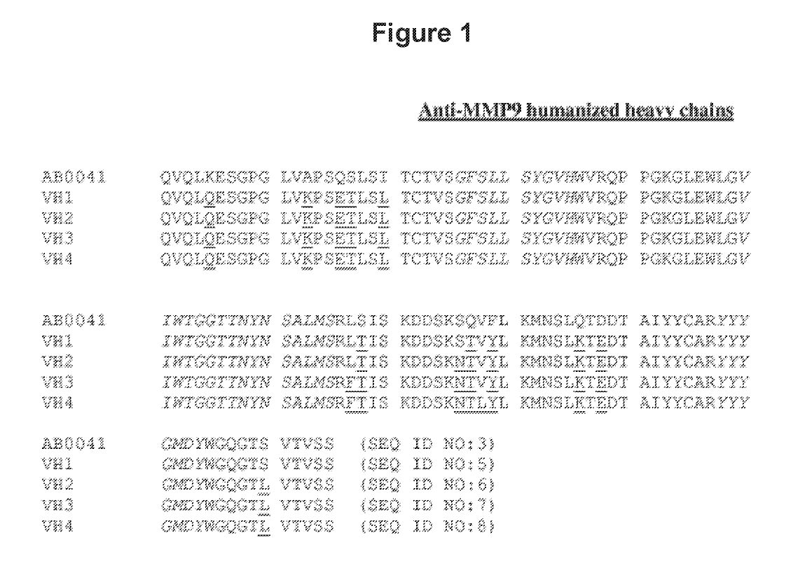

FIG. 1 shows an alignment of the amino acid sequences of the variable regions of the humanized heavy chains and indicates the differences in amino acid sequences in the framework regions among the four variants.

### Light-Chain Variants

Four light-chain variants were constructed, in a human kappa chain background, and are denoted Vk1, Vk2, Vk3 and Vk4. The amino acid sequences of their framework regions and CDRs are as follows:

Vk1:

(SEQ ID NO: 9)

DIVMTQSPSFLSASVGDRVTITCKASQDVRNTVAWYQQKTGKAPKLLIYS
SSYRNTGVPDRFTGSGSGTDFTLTISSLQAEDVAVYFCQQHYITPYTFGG
GTKVEIK;
Vk2:

(SEQ ID NO: 10)

DIVMTQSPSSLSASVGDRVTITCKASQDVRNTVAWYQQKPGKAPKLLIYS
SSYRNTGVPDRFTGSGSGTDFTLTISSLQAEDVAVYFCQQHYITPYTFGG
GTKVEIK;
Vk3:

(SEQ ID NO: 11)

DIQMTQSPSSLSASVGDRVTITCKASQDVRNTVAWYQQKPGKAPKLLIYS
SSYRNTGVPDRFSGSGSGTDFTLTISSLQAEDVAVYFCQQHYITPYTFGG
GTKVEIK;
Vk4:

(SEQ ID NO: 12)

DIQMTQSPSSLSASVGDRVTITCKASQDVRNTVAWYQQKPGKAPKLLIYS
SSYRNTGVPDRFSGSGSGTDFTLTISSLQAEDVAVYYCQQHYITPYTFGG
GTKVEIK.

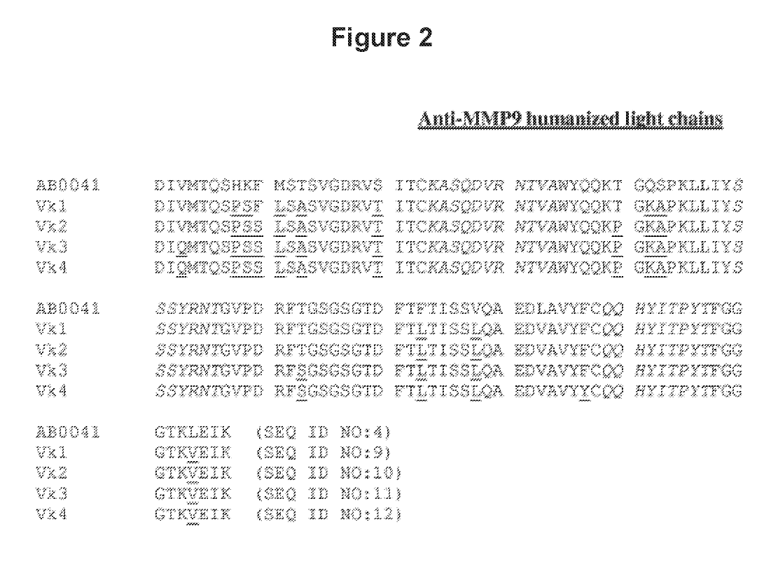

FIG. 2 shows an alignment of the amino acid sequences of the variable regions of the humanized light chains and indicates the differences in amino acid sequences in the framework regions among the four variants.

The humanized heavy and light chains are combined in all possible pair-wise combinations to generate a number of functional humanized anti-MMP9 antibodies. For example, provided are antibodies with a heavy chain variable (VH) region comprising the amino acid sequence set forth in any of SEQ ID NOs: 3, 5, 6, 7, and 8; antibodies comprising a light chain variable (VL) region having the amino acid sequence set forth in any of SEQ ID NOs: 4, 9, 10, 11, and 12; and antibodies with a heavy chain variable (VH) region comprising the amino acid sequence set forth in any of SEQ ID NOs: 3, 5, 6, 7, and 8 and a light chain variable (VL) region comprising the amino acid sequence set forth in any of SEQ ID NOs: 4, 9, 10, 11, and 12, as well as antibodies that compete for binding to MMP9 with such antibodies and antibodies comprising at least about 75%, 80%, 85%, 90%, 91%, 92%, 93%, 94%, 95%, 96%, 97%, 98%, 99% or more sequence identity with such antibodies. In one example, the antibody comprises a VH region with an amino acid sequence having at least about 75%, 80%, 85%, 90%, 91%, 92%, 93%, 94%, 95%, 96%, 97%, 98%, 99% or more sequence identity with SEQ ID NO: 7, and a VL region with an amino acid sequence having at least about 75%, 80%, 85%, 90%, 91%, 92%, 93%, 94%, 95%, 96%, 97%, 98%, 99% or more sequence identity with SEQ ID NO: 12, or a VH region of SEQ ID NO: 7 and a VL region of SEQ ID NO: 12.

Additional heavy chain variable region amino acid sequences comprising 75% or more, 80% or more, 90% or more, 95% or more, or 99% or more homology to the heavy chain variable region sequences disclosed herein are also provided. Furthermore, additional light chain variable region amino acid sequences comprising 75% or more, 80% or more, 90% or more, 95% or more, or 99% or more homology to the light chain variable region sequences disclosed herein are also provided.

Additional heavy chain variable region amino acid sequences comprising 75% or more, 80% or more, 90% or more, 95% or more, or 99% or more sequence identity to the heavy chain variable region sequences disclosed herein are also provided. Furthermore, additional light chain variable region amino acid sequences comprising 75% or more, 80% or more, 90% or more, 95% or more, or 99% or more sequence identity to the light chain variable region sequences disclosed herein are also provided.

### Complementarity-Determining Regions (CDRs)

In some embodiments, the CDRs of the heavy chain of exemplary provided anti-MMP9 antibodies or fragments thereof as disclosed herein comprise the following amino acid sequences:

CDR1:

(SEQ ID NO: 13)

GFSLLSYGVH;
CDR2:

(SEQ ID NO: 14)

VIWTGGTTNYNSALMS;
CDR3:

(SEQ ID NO: 15)

YYYGMDY.

Thus, among the provided anti-MMP9 antibodies or fragments thereof are antibodies or fragments thereof comprising a heavy chain CDR1 region with an amino acid sequence as set forth in SEQ ID NO: 13; antibodies or fragments thereof comprising a heavy chain CDR2 region with an amino acid sequence set forth in SEQ ID NO: 14; antibodies or fragments thereof comprising a heavy chain CDR3 region with an amino acid sequence as set forth in SEQ ID NO: 15; and antibodies or fragments thereof that compete for binding with or bind to the same epitope on MMP9 as such antibodies. In some cases, the antibodies of fragments thereof contain VH CDRs comprising the sequences set forth in SEQ ID NO: 13, 14, and 15.

In some embodiments, the CDRs of the light chain of exemplary anti-MMP9 antibodies or fragments thereof, as disclosed herein, comprise the following amino acid sequences:

CDR1:

(SEQ ID NO: 16)

KASQDVRNTVA;
CDR2:

(SEQ ID NO: 17)

SSSYRNT;
CDR3:

(SEQ ID NO: 18)

QQHYITPYT.

Thus, among the provided anti-MMP9 antibodies or fragments thereof are antibodies or fragments thereof comprising a light chain CDR1 region with an amino acid sequence as set forth in SEQ ID NO: 16; antibodies or fragments thereof comprising a light chain CDR2 region with an amino acid sequence set forth in SEQ ID NO: 17; antibodies or fragments thereof comprising a light chain CDR3 region with an amino acid sequence as set forth in SEQ ID NO: 18, and antibodies or fragments thereof that compete for binding with or bind to the same epitope on MMP9 as such antibodies. In some cases, the antibodies or fragments thereof, as disclosed herein, contain VL CDRs comprising the sequences set forth in SEQ ID NO: 16, 17, and 18.

An exemplary humanized variant anti-MMP9 antibody, AB0045 (humanized, modified IgG4 (S241P)) comprises the humanized AB0041 heavy chain variant VH3 comprising the sequence set forth in SEQ ID NO: 7:

QVQLQESGPGLVKPSETLSLTCTVSGFSLLSYGVHWVRQPPGKGLEWLGV
IWTGGTTNYNSALMSRFTISKDDSKNTVYLKMNSLKTEDTAIYYCARYYY
GMDYWGQGTLVTVSS;

and the humanized AB0041 light chain variant VH4 (comprising the light chain sequence set forth in Vk4 comprising the sequence set forth in SEQ ID NO: 12:

DIQMTQSPSSLSASVGDRVTITCKASQDVRNTVAWYQQKPGKAPKLLIYS
SSYRNTGVPDRFSGSGSGTDFTLTISSLQAEDVAVYYCQQHYITPYTFGG
GTKVEIK.

The AB0045 antibody comprises 1312 amino acids in length, is composed of two heavy chains and two light chains, and has a theoretical pI of about 7.90, extinction coefficient of about 1.50 AU/cm at 280 nm for 1 g/L, a molecular weight of about 144 kDa, and density of about 1 g/mL in formulation buffer (50-100 mg/mL product concentration).

The heavy chain of the AB0045 antibody comprises the sequence set forth in SEQ ID NO: 49:

MGWSLILLFLVAVATRVHSQVQLQESGPGLVKPSETLSLTCTVSGFSLLS
YGVHWVRQPPGKGLEWLGVIWTGGTTNYNSALMSRFTISKDDSKNTVYLK
MNSLKTEDTAIYYCARYYYGMDYWGQGTLVTVSSASTKGPSVFPLAPCSR
STSESTAALGCLVKDYFPEPVTVSWNSGALTSGVHTFPAVLQSSGLYSLS
SVVTVPSSSLGTKTYTCNVDHKPSNTKVDKRVESKYGPPCPPCPAPEFLG
GPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHY
TQKSLSLSLGK (signal sequence underlined; sequence
of the constant region presented italics);

and the light chain of the AB0045 antibody comprises the sequence set forth in SEQ ID NO: 50:

MRVPAQLLGLLLLWLPGARCDIQMTQSPSSLSASVGDRVTITCKASQDVR
NTVAWYQQKPGKAPKLLIYSSSYRNTGVPDRFSGSGSGTDFTLTISSLQA
EDVAVYYCQQHYITPYTFGGGTKVEIKRTVAAPSVFIFPPSDEQLKSGTA
SVVCLLNNFYPREAKVQWKVDNALQSGNSQESVTEQDSKDSTYSLSSTLT
LSKADYEKHKVYACEVTHQGLSSPVTKSFNRGEC (signal
sequence underlined; sequence of the constant
region presented italics).

The antibodies and fragments thereof disclosed herein further include those produced by the hybridoma designated M4, i.e., an antibody containing the heavy chain (IgG2b) sequence:

(SEQ ID NO: 30)

MAVLVLFLCLVAFPSCVLSQVQLKESGPGLVAPSQSLSITCTVSGFSLLS
YGVHWVRQPPGKGLEWLGVIWTGGSTNYNSALMSRLSISKDDSKSQVFLK
MNSLQTDDTAMYYCARYYYAMDYWGQGTSVTVSSAKTTPPSVYPLAPGCG
DTTGSSVTLGCLVKGYFPESVTVTWNSGSLSSSVHTFPALLQSGLYTMSS
SVTVPSSTWPSQTVTCSVAHPASSTTVDKKLEPSGPISTINPCPPCKECH
KCPAPNLEGGPSVFIFPPNIKDVLMISLTPKVTCVVVDVSEDDPDVRISW
FVNNVEVHTAQTQTHREDYNSTIRVVSALPIQHQDWMSGKEFKCKVNNKD
LPSPIERTISKIKGLVRAPQVYILPPPAEQLSRKDVSLTCLVVGFNPGDI
SVEWTSNGHTEENYKDTAPVLDSDGSYFIYSKLDIKTSKWEKTDSFSCNV
RHEGLKNYYLKKTISRSPGK (signal peptide set forth in
underlined text, variable region set forth in
plain text, and constant region set forth
in italics);

and the light chain (kappa) sequence:

(SEQ ID NO: 31)

MESQIQVFVFVFLWLSGVDGDIVMTQSHKFMFTSVGDRVSITCKASQDVR
NTVAWYQQKTGQSPKLLIYSASYRNTGVPDRFTGSISGTDFTFTISSVQA
EDLALYYCQQHYSTPYTFGGGTKLEVKRADAAPTVSIFPPSSEQLTSGGA
SVVCFLNNFYPKDINVKWKIDGSERQNGVLNSWTDQDSKDSTYSMSSTLT
LTKDEYERHNSYTCEATHKTSTSPIVKSFNRNEC (signal peptide
set forth in underlined text, variable region set
forth inplain text, and constant region set forth
in italics.

The M4 antibody comprises a variable heavy chain with an amino acid sequence:

(SEQ ID NO: 32)

QVQLKESGPGLVAPSQSLSITCTVSGFSLLSYGVHWVRQPPGKGLEWLGV
IWTGGSTNYNSALMSRLSISKDDSKSQVFLKMNSLQTDDTAMYYCARYYY
AMDYWGQGTSVTVSS (CDRs 1, 2, and 3 (SEQ ID NOs:
34, 35, and 36, respectively) underlined); and a
variable light chain with the amino acid sequence:

(SEQ ID NO: 33)

DIVMTQSHKFMFTSVGDRVSITCKASQDVRNTVAWYQQKTGQSPKLLIYS
ASYRNTGVPDRFTGSISGTDFTFTISSVQAEDLALYYCQQHYSTPYTFGG
GTKLEVK (CDRs 1, 2, and 3 (SEQ ID NOs: 37, 38, and
39, respectively) underlined).

The antibodies and fragments thereof disclosed herein further include those produced by the hybridoma designated M12, i.e., one with only a kappa chain, comprising the sequence:

(SEQ ID NO: 40)

QVFVYMLLWLSGVDGDIVMTQSQKFMSTSVGDRVSVTCKASQNVGTNVAW
YQQKPGQSPKALIYSASYRFSGVPDRFTGSGSGTDFTLTISNVQSEDLAE
YFCQQYNSYPYTFGGGTKLEIKRADAAPTVSIFPPSSEQLTSGGASVVCF
LNNFYPKDINVKWKIDGSERQNGVLNSWTDQDSKDSTYSMSSTLTLTKDE
YERHNSYTCEATHKTSTSPIVKSFNRNEC (signalpeptide set
forth in underlined text, variable region set
forth in plaintext, and constant region set forth
in it alics).

The M12 antibody comprises a variable light chain with the amino acid sequence:

(SEQ ID NO: 41)

DIVMTQSQKFMSTSVGDRVSVTCKASQNVGTNVAWYQQKPGQSPKALIYS
ASYRFSGVPDRFTGSGSGTDFTLTISNVQSEDLAEYFCQQYNSYPYTFGG
GTKLEIK (CDRs 1, 2,and 3 (SEQ ID NOs: 42, 43, and
44, respectively) underlined).

The antibodies and fragments thereof disclosed herein further include the mouse antibody designated AB0046, comprising a kappa light chain with an amino acid sequence:

(SEQ ID NO: 45)

MSSAQFLGLLLLCFQGTRCDIQMTQTTSSLSASLGDRVTISCSASQGISN
YLNWYQQKPDGTFKLLIYYTSILHSGVPSRFSGSGSGTDYSLTISNLEPE
DIATYYCQQYGWLPRTFGGGTKLEIKRADAAPTVSIFPPSSEQLTSGGAS
VVCFLNNFYPKDINVKWKIDGSERQNGVLNSWTDQDSKDSTYSMSSTLTL
TKDEYERHNSYTCEATHKTSTSPIVKSFNRNEC (signal peptide
set forth in underlined text, variable region set
forth in plain text, and constant region set forth
in italics);

and an IgG1 heavy chain with an amino acid sequence:

(SEQ ID NO: 46)

MGWSSIILFLVATATGVHSQVQLQQPGSVLVRPGASVKLSCTASGYTFTS
YWMNWVKQRPGQGLEWIGEIYPISGRTNYNEKFKVKATLTVDTSSSTAYM
DLNSLTSEDSAVYYCARSRANWDDYWGQGTTLTVSSAKTTPPSVYPLAPG
SAAQTNSMVTLGCLVKGYFPEPVTVTWNSGSLSSGVHTFPAVLQSDLYTL
SSSVTVPSSTWPSETVTCNVAHPASSTKVDKKIVPRDCGCKPCICTVPEV
SSVFIFPPKPKDVLTITLTPKVTCVVVDISKDDPEVQFSWFVDDVEVHTA
QTQPREEQFNSTFRSVSELPIMHQDWLNGKEFKCRVNSAAFPAPIEKTIS
KTKGRPKAPQVYTIPPPKEQMAKDKVSLTCMITDFFPEDITVEWQWNGQP
AENYKNTQPIMDTDGSYFVYSKLNVQKSNWEAGNTFTCSVLHEGLHNHHT
EKSLSHSPGK (signal peptide set forth in underlined
text, variable region set forth in plain text, and
constant region set forth in italics).

The following amino acid sequence comprises the framework regions and complementarity-determining regions (CDRs) of the variable region of the IgG1 heavy chain of AB0046 (with CDRs underlined):

(SEQ ID No: 47)

QVQLQQPGSVLVRPGASVKLSCTASGYTFTSYWMNWVKQRPGQGLEWIGE
IYPISGRTNYNEKFKVKATLTVDTSSSTAYMDLNSLTSEDSAVYYCARSR
ANWDDYWGQGTTLTVSS.

The following amino acid sequence comprises the framework regions and complementarity-determining regions (CDRs) of the variable region of the kappa light chain of AB0046 (with CDRs underlined):

(SEQ ID No: 48)

DIQMTQTTSSLSASLGDRVTISCSASQGISNYLNWYQQKPDGTFKLLIYY
TSILHSGVPSRFSGSGSGTDYSLTISNLEPEDIATYYCQQYGWLPRTFGG
GTKLEIK.

The antibodies or fragments thereof for use with the presently provided methods, compositions, and combinations can include any of the antibodies or fragments thereof described herein, including those comprising any combination of the various exemplified heavy and light chains, heavy and light chain variable regions, and CDRs.

### Nucleic Acids Encoding Anti-MMP9 Antibodies

The present disclosure provides nucleic acids encoding anti-MMP9 antibodies and functional fragments thereof. Accordingly, the present disclosure provides an isolated polynucleotide (nucleic acid) encoding an antibody or antigen-binding fragment as described herein, vectors containing such polynucleotides, and host cells and expression systems for transcribing and translating such polynucleotides into polypeptides.

The present disclosure also contemplates constructs in the form of plasmids, vectors, transcription or expression cassettes which comprise at least one polynucleotide as above.

The present disclosure also provides a recombinant host cell which comprises one or more constructs as above, as well as methods of production of the antibody or antigen-binding fragments thereof described herein, where one such method comprises expression of nucleic acid encoding a heavy chain polypeptide and a light chain polypeptide (in the same or different host cells, and from the same or different constructs) in a recombination host cell. Expression can be achieved by culturing under appropriate conditions recombinant host cells containing the nucleic acid. Following production by expression, an antibody or antigen-binding fragment can be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common bacterial host is E. coli.

Suitable vectors can be chosen or constructed, containing appropriate regulatory sequences, including operably linked promoter sequences, terminator sequences, polyadenylation sequences, enhancer sequences, marker genes and/or other sequences as appropriate. Vectors can be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference in their entirety.

The nucleic acid encoding a polypeptide of interest is integrated into the genome of the host cell or can be maintained as a stable or transient episomal element.

Any of a wide variety of expression control sequences—sequences that control the expression of a DNA sequence operatively linked to it—can be used in these vectors to express the DNA sequences. For example, a nucleic acid encoding a polypeptide of interest can be operably linked to a promoter, and provided in an expression construct for use in methods of production of recombinant MMP9 proteins or portions thereof.

Those of skill in the art are aware that nucleic acids encoding the antibody chains disclosed herein can be synthesized using standard knowledge and procedures in molecular biology.

The present disclosure provides, in some embodiments, isolated nucleic acids encoding the antibodies and fragments thereof disclosed herein, where the nucleic acids comprise a nucleotide sequence encoding a heavy chain polypeptide comprising CDRs with the amino acid sequences set forth in SEQ ID NOs: 13-15, and/or a light chain polypeptide comprising CDRs with the amino acid sequences set forth in SEQ ID NOs: 16-18. In one embodiment, the nucleotide sequence encodes the heavy chain polypeptide, which has an amino acid sequence selected from the group consisting of SEQ ID NOS: 1, 3, and 5-8. In another embodiment, the nucleotide sequence encodes the light chain polypeptide, which comprises an amino acid sequence selected from the group consisting of SEQ ID NOS: 2, 4, and 9-12. In yet another embodiment, the nucleotide sequence encodes the heavy chain polypeptide comprising a sequence selected from the group consisting of: SEQ ID NOs: 19-22, and/or a light chain polypeptide comprising a sequence selected from the group consisting of: SEQ ID NOs: 23-26.

In some embodiments, nucleotide sequences encoding the heavy and light chain amino acid sequences disclosed herein, are as follows:

VH1:
(SEQ ID NO: 19)
CAGGTGCAGC TGCAGGAATC CGGCCCTGGC CTGGTCAAGC CCTCCGAGAC ACTGTCCCTG ACCTGCACCG
TGTCCGGCTT CTCCCTGCTG TCCTACGGCG TGCACTGGGT CCGACAGCCT CCAGGGAAGG GCCTGGAATG
GCTGGGCGTG ATCTGGACCG GCGGCACCAC CAACTACAAC TCCGCCCTGA TGTCCCGGCT GACCATCTCC
AAGGACGACT CCAAGTCCAC CGTGTACCTG AAGATGAACT CCCTGAAAAC CGAGGACACC GCCATCTACT
ACTGCGCCCG GTACTACTAC GGCATGGACT ACTGGGGCCA GGGCACCTCC GTGACCGTGT CCTCA;
VH2:
(SEQ ID NO: 20)
CAGGTGCAGC TGCAGGAATC CGGCCCTGGC CTGGTCAAGC CCTCCGAGAC ACTGTCCCTG ACCTGCACCG
TGTCCGGCTT CTCCCTGCTG TCCTACGGCG TGCACTGGGT CCGACAGCCT CCAGGCAAAG GCCTGGAATG
GCTGGGCGTG ATCTGGACCG GCGGCACCAC CAACTACAAC TCCGCCCTGA TGTCCCGGCT GACCATCTCC
AAGGACGACT CCAAGAACAC CGTGTACCTG AAGATGAACT CCCTGAAAAC CGAGGACACC GCCATCTACT
ACTGCGCCCG GTACTACTAC GGCATGGACT ACTGGGGCCA GGGCACCCTG GTCACCGTGT CCTCA;
VH3:
(SEQ ID NO: 21)
CAGGTGCAGC TGCAGGAATC CGGCCCTGGC CTGGTCAAGC CCTCCGAGAC ACTGTCCCTG ACCTGCACCG
TGTCCGGCTT CTCCCTGCTG TCCTACGGCG TGCACTGGGT CCGACAGCCT CCAGGCAAAG GCCTGGAATG
GCTGGGCGTG ATCTGGACCG GCGGCACCAC CAACTACAAC TCCGCCCTGA TGTCCCGGTT CACCATCTCC
AAGGACGACT CCAAGAACAC CGTGTACCTG AAGATGAACT CCCTGAAAAC CGAGGACACC GCCATCTACT
ACTGCGCCCG GTACTACTAC GGCATGGACT ACTGGGGCCA GGGCACCCTG GTCACCGTGT CCTCA;
VH4:
(SEQ ID NO: 22)
CAGGTGCAGC TGCAGGAATC CGGCCCTGGC CTGGTCAAGC CCTCCGAGAC ACTGTCCCTG ACCTGCACCG
TGTCCGGCTT CTCCCTGCTG TCCTACGGCG TGCACTGGGT CCGACAGCCT CCAGGCAAAG GCCTGGAATG
GCTGGGCGTG ATCTGGACCG GCGGCACCAC CAACTACAAC TCCGCCCTGA TGTCCCGGTT CACCATCTCC
AAGGACGACT CCAAGAACAC CCTGTACCTG AAGATGAACT CCCTGAAAAC CGAGGACACC GCCATCTACT
ACTGCGCCCG GTACTACTAC GGCATGGACT ACTGGGGCCA GGGCACCCTG GTCACCGTGT CCTCA;
Vk1:
(SEQ ID NO: 23)
GACATCGTGA TGACCCAGTC CCCCAGCTTC CTGTCCGCCT CCGTGGGCGA CAGAGTGACC ATCACATGCA
AGGCCTCTCA GGACGTGCGG AACACCGTGG CCTGGTATCA GCAGAAAACC GGCAAGGCCC CCAAGCTGCT
GATCTACTCC TCCTCCTACC GGAACACCGG CGTGCCCGAC CGGTTTACCG GCTCTGGCTC CGGCACCGAC
TTTACCCTGA CCATCAGCTC CCTGCAGGCC GAGGACGTGG CCGTGTACTT CTGCCAGCAG CACTACATCA
CCCCCTACAC CTTCGGCGGA GGCACCAAGG TGGAAATAAA A;
Vk2:
(SEQ ID NO: 24)
GACATCGTGA TGACCCAGTC CCCCTCCAGC CTGTCCGCCT CTGTGGGCGA CAGAGTGACC ATCACATGCA
AGGCCTCTCA GGACGTGCGG AACACCGTGG CCTGGTATCA GCAGAAGCCC GGCAAGGCCC CCAAGCTGCT
GATCTACTCC TCCTCCTACC GGAACACCGG CGTGCCCGAC CGGTTTACCG GCTCTGGCTC CGGCACCGAC
TTTACCCTGA CCATCAGCTC CCTGCAGGCC GAGGACGTGG CCGTGTACTT CTGCCAGCAG CACTACATCA
CCCCCTACAC CTTCGGCGGA GGCACCAAGG TGGAAATAAA A;
Vk3:
(SEQ ID NO: 25)
GACATCCAGA TGACCCAGTC CCCCTCCAGC CTGTCCGCCT CTGTGGGCGA CAGAGTGACC ATCACATGCA
AGGCCTCCCA GGACGTGCGG AACACCGTGG CCTGGTATCA GCAGAAGCCC GGCAAGGCCC CCAAGCTGCT
GATCTACTCC TCCTCCTACC GGAACACCGG CGTGCCCGAC CGGTTCTCTG GCTCTGGAAG CGGCACCGAC
TTTACCCTGA CCATCAGCTC CCTGCAGGCC GAGGACGTGG CCGTGTACTT CTGCCAGCAG CACTACATCA
CCCCCTACAC CTTCGGCGGA GGCACCAAGG TGGAAATAAA A;
Vk4:
(SEQ ID NO: 26)
GACATCCAGA TGACCCAGTC CCCCTCCAGC CTGTCCGCCT CTGTGGGCGA CAGAGTGACC ATCACATGCA
AGGCCTCTCA GGACGTGCGG AACACCGTGG CCTGGTATCA GCAGAAGCCC GGCAAGGCCC CCAAGCTGCT
GATCTACTCC TCCTCCTACC GGAACACCGG CGTGCCCGAC CGGTTCTCTG GCTCTGGAAG CGGCACCGAC
TTTACCCTGA CCATCAGCTC CCTGCAGGCC GAGGACGTGG CCGTGTACTA CTGCCAGCAG CACTACATCA
CCCCCTACAC CTTCGGCGGA GGCACCAAGG TGGAAATAAA A.

Because the structure of antibodies and fragments thereof, including the juxtaposition of CDRs and framework regions in the variable region, the structure of framework regions and the structure of heavy- and light-chain constant regions, is well-known in the art, it is well within the skill of the art to obtain related nucleic acids that encode anti-MMP-9 antibodies. Accordingly, polynucleotides comprising nucleic acid sequences having at least 75%, at least 80%, at least 85%, at least 90%, at least 95%, at least 98% and at least 99% homology to any of the nucleotide sequences disclosed herein are also provided. In one example, the polynucleotide contains at least about 75%, 80%, 85%, 90%, 91%, 92%, 93%, 94%, 95%, 96%, 97%, 98%, 99% or more sequence identity with SEQ ID NO: 21, or is SEQ ID NO: 21; and/or contains at least about 75%, 80%, 85%, 90%, 91%, 92%, 93%, 94%, 95%, 96%, 97%, 98%, 99% or more sequence identity with SEQ ID NO: 26 or is SEQ ID NO: 26.

### Treatment Methods and Uses

Filgotinib and MMP9 binding proteins may be used in combination therapies. Accordingly, the present disclosure provides methods for treating inflammatory disorders in a human in need thereof, comprising administering to the human a therapeutically effective amount of filgotinib or a pharmaceutically acceptable salt, solvate, polymorph, or metabolite thereof, and a therapeutically effective amount of an MMP9 binding protein. The inflammatory disorders include, but are not limited to, rheumatoid arthritis, Crohn's disease, osteoarthritis, allergic airway disease, multiple sclerosis, inflammatory bowel disease, sepsis, psoriasis, misregulated TNF expression, and graft rejection.

As used herein, “treating” and “treatment” of a disease include the following: (1) preventing or reducing the risk of developing the disease, i.e., causing the clinical symptoms of the disease not to develop in a subject that may be exposed to or predisposed to the disease but does not yet experience or display symptoms of the disease, (2) inhibiting the disease, i.e., arresting or reducing the development of the disease or its clinical symptoms, and (3) relieving the disease, i.e., causing regression of the disease or its clinical symptoms.

“Subject” and “subjects” refers to humans, domestic animals (e.g., dogs and cats), farm animals (e.g., cattle, horses, sheep, goats and pigs), laboratory animals (e.g., mice, rats, hamsters, guinea pigs, pigs, rabbits, dogs, and monkeys), and the like.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art. The selected compounds may be further characterized to examine the safety or tolerance dosage in human or non-human subjects. Such properties may be examined using commonly known methods to those skilled in the art.

In general, MMP9 binding proteins, e.g., the AB0045 antibody or any anti-MMP9 antibody or fragment thereof disclosed herein, are administered in a therapeutically effective amount, e.g., in an amount inhibit MMP9 activity, and/or to treat an inflammatory disorder. Similarly, filgotinib is generally administered in a therapeutically effective amount, e.g., in an amount to treat an inflammatory disorder.

As used herein, unless otherwise specified, the term “therapeutically effective amount” or “effective amount” refers to an amount of an agent or compound or composition that when administered (either alone or in combination with another therapeutic agent, as may be specified) to a subject is effective to prevent or ameliorate the disease condition or the progression of the disease, or result in amelioration of symptoms, e.g., treatment, healing, prevention or amelioration of the relevant medical condition, or an increase in rate of treatment, healing, prevention or amelioration of such conditions. For an individual active ingredient administered alone, a therapeutically effective dose refers to that ingredient alone. For a combination of active ingredients, a therapeutically effective dose refers to combined amounts of the active ingredients that result in the therapeutic effect, whether administered in combination, serially or simultaneously.

An antibody or fragment thereof, as disclosed herein, may be administered as part of a combination therapy with filgotinib. Accordingly, the present disclosure provides a method for treating an inflammatory disorder in a human in need thereof, comprising administering to the human: a therapeutically effective amount of filgotinib or a pharmaceutically acceptable salt, solvate, polymorph, or metabolite thereof, and a therapeutically effective amount of an antibody or fragment thereof, as disclosed herein, such as a selective anti-MMP9 antibody. In some embodiments, filgotinib, or a pharmaceutically acceptable salt, solvate, polymorph, or metabolite thereof, is administered prior to, after or concurrently with the antibody or fragment thereof, as disclosed herein.

In some embodiments, combination therapy with filgotinib or a pharmaceutically acceptable salt, solvate, polymorph, or metabolite thereof, and an anti-MMP9 antibody or fragment thereof, as disclosed herein, provides synergy. In some embodiments, the amount or dosage of filgotinib or a pharmaceutically acceptable salt, solvate, polymorph, or metabolite thereof, and the antibody or fragment thereof, used in combination, does not exceed the level at which each agent is used individually, e.g., as a monotherapy. In certain embodiments, the amount or dosage of filgotinib, or a pharmaceutically acceptable salt, solvate, polymorph, or metabolite thereof, and the amount or dosage of the antibody or fragment thereof, used in combination, is lower (e.g., at least 20%, at least 30%, at least 40%, or at least 50% lower) than the amount or dosage of each agent used individually, e.g., as a monotherapy. In other embodiments, the amount or dosage of filgotinib, or a pharmaceutically acceptable salt, solvate, polymorph, or metabolite thereof, and the amount or dosage of the antibody or fragment thereof, used in combination that results in treatment of an inflammatory disorder is lower (e.g., at least 20%, at least 30%, at least 40%, or at least 50% lower) than the amount or dosage of each agent used individually, e.g., as a monotherapy.

In some embodiments, filgotinib, or a pharmaceutically acceptable salt, solvate, polymorph, or metabolite thereof, is administered intravenously, intramuscularly, parenterally (e.g., via an intravenous, intramuscular, inter-arterial, or subcutaneous route), nasally or orally. In some embodiments, the antibody or fragment thereof, as disclosed herein, is administered parenterally (e.g., via an intravenous, intramuscular, inter-arterial, or subcutaneous route), nasally or orally. In some embodiments, filgotinib, or a pharmaceutically acceptable salt, solvate, polymorph, or metabolite thereof, is administered intravenously, intramuscularly, parenterally, nasally or orally prior to, after, or concurrently with the intravenous, intramuscular, parenteral, nasal or oral administration of the antibody or fragment thereof, as disclosed herein.

In some embodiments, the antibody or fragment thereof, as disclosed herein, is administered alone, as a monotherapy for treatment of an inflammatory disorder. In some embodiments, filgotinib, or a pharmaceutically acceptable salt, solvate, polymorph, or metabolite thereof, is administered alone as a monotherapy for treatment of an inflammatory disorder.

In some embodiments, filgotinib or a pharmaceutically acceptable salt, solvate, polymorph, or metabolite thereof, and/or an antibody or fragment thereof, as disclosed herein, may be administered with other immunotherapeutic agents, including antibodies against LOXL2 (lysyl oxidase-like 2) and/or DDR1 (discoidin domain receptor 1), for treatment of an inflammatory disorder.

In some embodiments, the methods and compositions disclosed herein are used to treat a disorder causally related or attributable to aberrant activity of JAK (e.g., aberrant activity of JAK1 and/or JAK2), including inflammatory conditions, autoimmune diseases, proliferative diseases, transplantation rejection, diseases involving impairment of cartilage turnover, congenital cartilage malformations, and diseases associated with hypersecretion of IL6.

In some embodiments, the methods and compositions disclosed herein are used to treat a disorder that is causally related or attributable to MMP9 expression. Expression of matrix metalloproteinases (MMPs), and MMP9 in particular, is associated with a variety of disease pathologies, including inflammatory an autoimmune diseases. MMP9 can promote disease through its destructive remodeling of basement membrane and other structural proteins, and/or by increasing vascular permeability and bioavailability of growth factors and cytokines such as TGFβ, VEGF, TNFα, IL-6, and IL-1(3. MMP9 regulates the bioavailability of ECM-sequestered VEGF and FGF-2, as well as membrane-tethered EGF. In some aspects, the methods and compositions disclosed herein inhibit MMP9 without affecting other MMPs, such as MMP2.

In some embodiments, the present disclose provides a method for treating an inflammatory disorder in a human in need thereof, comprising administering to the human: (i) a therapeutically effective amount of filgotinib or a pharmaceutically acceptable salt, solvate, polymorph, or metabolite thereof; and (ii) a therapeutically effective amount of a selective anti-matrix metalloproteinase 9 (MMP9) antibody.

In one embodiment, the selective anti-MMP9 antibody selectively binds human MMP9 protein and does not bind other human MMP proteins. In one embodiment, the selective anti-MMP9 antibody binds MMP9 outside the catalytic domain.

In one embodiment, the selective anti-MMP9 antibody inhibits the enzymatic activity of MMP9. In one embodiment, the inhibition of the enzymatic activity is non-competitive.

In one embodiment, the selective anti-MMP9 antibody is human antibody or a humanized antibody.

In one embodiment, the selective anti-MMP9 antibody comprises a heavy chain variable (VH) region comprising a heavy chain complementary determining region (CDR) with an amino acid sequence selected from the group consisting of SEQ ID NO: 13, SEQ ID NO: 14, SEQ ID NO: 15, and combinations thereof. In one embodiment, the VH region comprises a heavy chain CDR1 with the amino acid sequence of SEQ ID NO: 13, a heavy chain CDR2 with the amino acid sequence of SEQ ID NO: 14, and a heavy chain CDR3 with the amino acid sequence of SEQ ID NO: 15.

In one embodiment, the selective anti-MMP9 antibody comprises a light chain variable (VL) region comprising a light chain complementary determining region (CDR) with an amino acid sequence selected from the group consisting of SEQ ID NO: 16, SEQ ID NO: 17, SEQ ID NO: 18, and combinations thereof. In one embodiment, the VL region comprises a light chain CDR1 with the amino acid sequence of SEQ ID NO: 16, a light chain CDR2 with the amino acid sequence of SEQ ID NO: 17, and a light chain CDR3 with the amino acid sequence of SEQ ID NO: 18.

In one embodiment, the VH region of the selective anti-MMP9 antibody comprises the amino acid sequence set forth in SEQ ID NO: 1, SEQ ID NO: 3, SEQ ID NO: 5, SEQ ID NO: 6, SEQ ID NO: 7, or SEQ ID NO: 8.

In one embodiment, the VL region of the selective anti-MMP9 antibody comprises the amino acid sequence set forth in SEQ ID NO: 2, SEQ ID NO: 4, SEQ ID NO: 9, SEQ ID NO: 10, SEQ ID NO: 11, or SEQ ID NO: 12.

In one embodiment, the VH region of the selective anti-MMP9 antibody comprises the amino acid sequence set forth in SEQ ID NO: 7 and the VL region comprises the amino acid sequence set forth in SEQ ID NO: 12.

In one embodiment, the selective anti-MMP9 antibody comprises a VH region with the amino acid sequence set forth in SEQ ID NO: 7, and a VL region with the amino acid sequence set forth in SEQ ID NO: 12.

In one embodiment, the selective anti-MMP9 antibody specifically binds to an epitope of MMP9, wherein the epitope comprises an amino acid residue within a region of MMP9, the region consisting of residues 104-119, residues 159-166, or residues 191-202 of SEQ ID NO: 27. In one embodiment, the epitope comprises E111, D113, R162, or 1198 of SEQ ID NO: 27. In one embodiment, the epitope comprises R162 of SEQ ID NO: 27.

In one embodiment, the (i) filgotinib or a pharmaceutically acceptable salt, solvate, polymorph, or metabolite thereof, and (ii) the selective anti-MMP9 antibody are conjugated to form an antibody-drug conjugate. In one embodiment, the conjugate further comprises a linker between (i) and (ii). In one embodiment, the linker is cleavable or non-cleavable in an intracellular environment.

In some embodiments, the present disclosure provides a method for treating an inflammatory disorder in a human in need thereof, comprising administering to the human a co-formulation of: (i) filgotinib or a pharmaceutically acceptable salt, solvate, polymorph, or metabolite thereof; (ii) a selective anti-MMP9 antibody, as disclosed herein, and (iii) a pharmaceutically acceptable carrier. In one embodiment, the co-formulation is administered parenterally, nasally or orally.

In some embodiments, the present disclosure provides a method for treating cancer in a human in need thereof, comprising administering to the human an antibody-drug conjugate of: (i) filgotinib or a pharmaceutically acceptable salt, solvate, polymorph, or metabolite thereof; (ii) a selective anti-MMP9 antibody, as disclosed herein, and (iii) a pharmaceutically acceptable carrier.

In some embodiments, the present disclosure provides use of a composition for the manufacture of a medicament for treating an inflammatory disorder, the composition comprising: (i) a therapeutically effective amount of filgotinib or a pharmaceutically acceptable salt, solvate, polymorph, or metabolite thereof; and (ii) a therapeutically effective amount of a selective anti-MMP9 antibody, as disclosed herein.

In some embodiments, the present disclosure in another embodiment use of a composition for the manufacture of a medicament for treating inflammatory bowel disease, Crohn's disease, or rheumatoid arthritis, the composition comprising: (i) a therapeutically effective amount of filgotinib or a pharmaceutically acceptable salt, solvate, polymorph, or metabolite thereof; and (ii) a therapeutically effective amount of a selective anti-MMP9 antibody, as disclosed herein.

In some embodiments, the treatment methods include steps for monitoring treatment, including for monitoring efficacy or activity, such as pharmacodynamic activity. In some examples, such methods include detecting or measuring the presence, absence, levels, and/or expression of markers, such as cytokines and other inflammatory markers that are indicative of efficacy of treatment, in biological test samples obtained from subjects being treated using the methods and compositions. The samples typically are blood samples or serum samples but can include other biological samples as described herein. Among the markers for use in such methods are Tissue Inhibitor of Metalloproteinases 1 (TIMP-1), Tumor Necrosis Factor alpha (TNF-alpha), Macrophage Inflammatory Protein-2 (MIP-2), Interleukin-17A (IL-17A), CXCL10, Lymphotactin, Macrophage Inflammatory Protein-1 beta (MIP-1 beta), Oncostatin-M (OSM), Interleukin-6 (IL-6), Monocyte Chemotactic Protein 3 (MCP-3), Vascular Endothelial Growth Factor A (VEGF-A), Monocyte Chemotactic Protein-5 (MCP-5), Interleukin-1 alpha (IL-1 alpha), Macrophage Colony-Stimulating Factor-1 (M-CSF-1), Myeloperoxidase (MPO), Growth-Regulated Alpha Protein (KC/GRO), Interleukin-7 (IL-7), Leukemia Inhibitory Factor (LIF), Apolipoprotein A-I (Apo A-I), C-Reactive Protein (CRP), Granulocyte Chemotactic Protein-2 (GCP-2), Interleukin-11 (IL-11), Monocyte Chemotactic Protein 1 (MCP-1), von Willebrand factor (vWF), and Stem Cell Factor (SCF) gene products. In some embodiments, the markers are selected from among KC/GRO, LIF, CXCL10, MPO, MIP-2, and MCP-5 gene products, for example, when the disease is IBD, such as UC.

In some embodiments, after each therapeutic cycle, the patients are monitored for the levels of MMP9 antibodies, MMP9, or other suitable biomarkers.

Among the provided methods are those that provide improved safety profiles compared to available treatments and therapeutic regimens and/or sustained long-term efficacy in treating such diseases and conditions.

### Subject

The human in need thereof may be an individual who has or is suspected of having an inflammatory disorder. In some embodiments, the human is at risk of developing an inflammatory disorder (e.g., a human who is genetically or otherwise predisposed to developing an inflammatory disorder) and who has or has not been diagnosed with the inflammatory disorder. As used herein, an “at risk” subject is a subject who is at risk of developing an inflammatory disorder. The subject may or may not have detectable disease, and may or may not have displayed detectable disease prior to the treatment methods described herein. An at risk subject may have one or more so-called risk factors, which are measurable parameters that correlate with development of an inflammatory disorder, such as described herein. A subject having one or more of these risk factors has a higher probability of developing an inflammatory disorder than an individual without these risk factor(s).

These risk factors may include, for example, age, sex, race, diet, history of previous disease, presence of precursor disease, genetic (e.g., hereditary) considerations, and environmental exposure. In some embodiments, a human at risk for an inflammatory disorder includes, for example, a human whose relatives have experienced this disease, and those whose risk is determined by analysis of genetic or biochemical markers. Prior history of having an inflammatory disorder may also be a risk factor for instances of recurrence thereof.

In some embodiments, provided herein is a method for treating a human who exhibits one or more symptoms associated with an inflammatory disorder. The human may be at various stages (e.g., an early stage, an advanced stage, etc.) of the inflammatory disorder.

In some embodiments, provided herein is a method for treating a human who is undergoing one or more standard therapies for treating an inflammatory disorder. Thus, in some foregoing embodiments, the combination of filgotinib or a pharmaceutically acceptable salt, solvate, polymorph, or metabolite thereof, and an antibody or fragment thereof, as disclosed herein, may be administered before, during, or after administration of such standard therapies.

### Inflammatory Disorders

In some embodiments, the methods and compositions (conjugates and/or co-formulations) disclosed herein are used in the treatment of an inflammatory disorder. Among the inflammatory disorders are inflammatory bowel disease (IBD) (including Crohn's disease, ulcerative colitis (UC), and indeterminate colitis), collagenous colitis, rheumatoid arthritis, osteoarthritis, septicemia, sepsis, psoriasis, myestenia gravis, acute disseminated encephalomyelitis, idiopathic thrombocytopenic purpura, Sjoegren's syndrome, autoimmune hemolytic anemia, multiple sclerosis, muscular dystrophy, lupus, allergy, asthma, chronic obstructive pulmonary disease (COPD), non-alcoholic steatohepatitis (NASH), and metabolic disorders characterized by impaired insulin production and glucose intolerance (e.g., Insulin Dependent Diabetes Mellitus (IDDM, also known as type 1 diabetes), and Non-Insulin-Dependent Diabetes Mellitus (NIDDM, also known as type 2 diabetes)).

In some embodiments, the inflammatory condition is selected from rheumatoid arthritis, osteoarthritis, allergic airway disease (e.g. asthma), and inflammatory bowel diseases. In one embodiment, the inflammatory condition is rheumatoid arthritis. In one embodiment, the inflammatory condition is inflammatory bowel disease. In one embodiment, the inflammatory disorders is Crohn's disease.

In one embodiment, the methods and compositions disclosed herein are used to treat an autoimmune disease such as COPD, asthma, systemic lupus erythematosis, and type I diabetes mellitus.

In another embodiment, the methods and compositions disclosed herein are used to treat transplantation rejection, such as organ transplant rejection.

In additional embodiments, the methods and compositions disclosed herein are used to treat a disease involving impairment of cartilage turnover.

In further embodiments, the methods and compositions disclosed herein are used to treat congenital cartilage malformation.

In various embodiments, the methods and compositions disclosed herein are used to treat a disease associated with hypersecretion of IL6, in particular Castleman's disease or mesangial proliferative glomerulonephritis.

In some embodiments, the methods and compositions disclosed herein protect against or reduce tissue injury, systemic inflammation, and/or local inflammation in a subject having such a disease or condition; in some examples, both tissue injury and inflammation are treated by the methods.

In certain embodiments, the methods and compositions disclosed herein are associated with reduced toxicity and/or reduced induction of musculoskeletal syndrome (MSS) or similar symptoms, compared to that observed with pan-MMP inhibitors, such as marimastat.

In various embodiments, the subject has had an inadequate response to another therapy for the inflammatory disorder, such as to an anti-TNF antibody (e.g., infliximab). Thus, among the provided methods and compositions are those effective at treating inflammation in such subjects.

### Rheumatoid Arthritis

Rheumatoid Arthritis (RA) is a chronic inflammatory and degenerative joint disease affecting about 1% of the worldwide adult population, with a higher prevalence in women. Although RA can occur at any age, it usually begins between the ages of 40 and 60. In particular, elderly patients are at high risk for adverse events from drug-drug interactions (DDIs) due to chronic disease, physiologic changes associated with aging, and the tendency to use multiple medications. The average older person uses two to six prescription medications and one to three non-prescription medications on a routine basis. The most common mechanism underlying DDI relates to the interplay with cytochrome P450 enzymes (CYP450s), with the inhibition of these enzymes being most often responsible for life-threatening interactions. In addition to these metabolic enzymes, the role drug transporters play in DDI, safety, and effectiveness of drugs has been greatly appreciated in recent years. Transporter-inhibiting drugs, such as filgotinib, or a pharmaceutically acceptable salt, solvate, polymorph, or metabolite thereof, can alter the transporter functional activity and/or protein expression, hence causing transporter-specific interactions.

Matrix metalloproteinase-9 (MMP9) is induced in the serum, synovial fluid, and synovium of RA patients, and the MMP9/TIMP-1 ratio is altered in favor of increased proteolytic activity. MMP9 is secreted by disease-mediating osteoclasts and activated cells of the monocyte/macrophage lineage. Resistance to antibody-induced arthritis disease phenotypes is observed in a MMP9 knock-out mouse strain. MMP9 degrades the unwound collagen II created by the cleavage activity of collagenases, such as MMP8, and thereby contributes to the destruction of articular cartilage.

Accordingly, in some embodiments, the methods and compositions disclosed herein are used to treat RA. In one such embodiment, the present disclose provides a method for treating rheumatoid arthritis in a human in need thereof, comprising administering to the human: (i) a therapeutically effective amount of filgotinib or a pharmaceutically acceptable salt, solvate, polymorph, or metabolite thereof; and (ii) a therapeutically effective amount of an antibody or fragment thereof, as disclosed herein, such as a selective anti-MMP9 antibody.

### Inflammatory Bowel Diseases

Inflammatory bowel diseases (IBDs) as used herein is a collective term describing inflammatory disorders of the gastrointestinal tract, the most common forms of which are ulcerative colitis and Crohn's disease. Other forms of IBD that can be treated with the presently disclosed compounds, compositions and methods include diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, Behçet's disease, gastroduodenal CD, jejunoileitis, ileitis, ileocolitis, Crohn's (granulomatous) colitis, irritable bowel syndrome, mucositis, radiation induced enteritis, short bowel syndrome, celiac disease, stomach ulcers, diverticulitis, pouchitis, proctitis, and chronic diarrhea.

Treating or treatment of IBD includes: (1) preventing or reducing the risk of developing IBD, i.e., causing the clinical symptoms of IBD not to develop in a subject that may be exposed to, or predisposed to, the disease but does not yet experience or display symptoms of IBD, (2) inhibiting the disease, i.e., arresting or reducing the development of IBD, or its clinical symptoms, and (3) relieving IBD, i.e., causing regression of IBD, or its clinical symptoms. Symptoms of IBD refer to detected symptoms including, but not limited to, abdominal pain, diarrhea, rectal bleeding, weight loss, fever, loss of appetite, and other more serious complications, such as dehydration, anemia and malnutrition. A number of such symptoms are subject to quantitative analysis (e.g., weight loss, fever, anemia, etc.). Some symptoms are readily determined from a blood test (e.g., anemia) or a test that detects the presence of blood (e.g., rectal bleeding). Reducing symptoms, such as symptoms of IBD, refers to a qualitative or quantitative reduction in detectable symptoms, including but not limited to a detectable impact on the rate of recovery from disease (e.g., rate of weight gain). The diagnosis is typically determined by way of an endoscopic observation of the mucosa, and pathologic examination of endoscopic biopsy specimens.

The course of IBD varies, and is often associated with intermittent periods of disease remission and disease exacerbation. Various methods have been described for characterizing disease activity and severity of IBD, as well as response to treatment in subjects having IBD. Treatment according to the presently disclosed methods is generally applicable to a subject having IBD of any level or degree of disease activity.

The presently disclosed treatment methods can also be applied at any point in the course of the disease. In certain embodiments, the methods disclosed herein are applied to a subject having IBD during a time period of remission (i.e., inactive disease). In such embodiments, the present methods provide benefit by extending the time period of remission (e.g., extending the period of inactive disease) or by preventing, reducing, or delaying the onset of active disease. In other embodiments, the methods disclosed herein may be applied to a subject having IBD during a period of active disease. Such methods provide benefit by reducing the duration of the period of active disease, reducing or ameliorating one or more symptoms of IBD, or treating IBD.

Measures for determining efficacy of treatment of IBD in clinical practice have been described and include, for example, the following: symptom control; fistula closure; extent of corticosteroid therapy required; and, improvement in quality of life. Heath-related quality of life (HRQL) can be assessed using the Inflammatory Bowel Disease Questionnaire (IBDQ), which is extensively used in clinical practice to assess quality of life in a subject with IBD. Improvements in any of the foregoing response criteria are specifically provided by the methods of the present disclosure.

As indicated above, ulcerative colitis (UC) is one of the two major IBDs, characterized by diffuse mucosal inflammation, and associated ulceration, of the colon. The chronic course of UC includes intermittent disease exacerbations followed by periods of remission. Many patients experience insufficient response to agents such as anti-TNFα targeted therapeutics and continue to suffer from disease-related symptoms. Patients with UC have a significantly elevated risk of colon cancer after 8-10 years of disease activity. Inflammatory bowel disease (IBD) therapeutics can modulate disease by preventing recruitment and access of inflammatory cells to the disease site, preventing activation of cells at the disease site, and/or inhibiting the downstream effects of cell activation.

Crohn's disease (CD) is a chronic inflammatory disorder of the gastrointestinal tract defined by relapsing and remitting episodes, with progression to complications such as fistula formation, abscesses, or strictures. Extraintestinal manifestations such as uveitis, arthritis, skin lesions, and kidney stones occur in upwards of 40% of patients. The treatment paradigm for mild-to-moderate Crohn's has been antibiotics such as ciprofloxacin and flagyl, 5-ASAs, budesonide, or systemic corticosteroids, however, the long-term side effects of systemic steroids greatly dampens their utility. Patients with mild-to-moderate disease who fail these first line therapies are often placed on the on azathioprine remain in remission at one-year. For patients who fail azathioprine or those with more severe disease, TNF-α blockade with agents such as infliximab remain the last option. As opposed to UC where surgical resection is curative, such therapy is more difficult for Crohn's patients for two reasons: 1) disease is diffuse throughout the GI tract and in instances of isolated disease (e.g., terminal ileum), resection is frequently associated with recurrent disease at the site of the resection 2) since the disease is transmural, surgical resection places patients at risk for future stricture and/or fistula development.

In some embodiments, the subject has moderate to severe CD, e.g., has severe CD. In some embodiments, the subject has steroid dependent CD. In some aspects, the treatment methods replace or are administered as an alternative to corticosteroid treatment.

In some embodiments, the subject has been non-responsive to other CD therapies, such as TNF antagonists, such as anti-TNF antibodies (such as infliximab and/or adalimumab), i.e., TNF antagonist-refractory patients. For example, in some embodiments, the subject is a patient who has failed to achieve long-term remission on infliximab therapy or other TNF-alpha targeting treatment. In other cases, the subject has been non-responsive to another CD therapy. In some aspects, the methods provide treatment with an improved safety protocol as compared to such treatments, or provide treatment with more sustained, long-term efficacy.

In some embodiments, the present disclose thus provides a method for treating CD in a human in need thereof, comprising administering to the human: (i) a therapeutically effective amount of filgotinib or a pharmaceutically acceptable salt, solvate, polymorph, or metabolite thereof; and (ii) a therapeutically effective amount of an antibody or fragment thereof, as disclosed herein, such as a selective anti-MMP9 antibody.

### Conjugates, Compositions, and Modes of Administration

In some embodiments, the filgotinib or a pharmaceutically acceptable salt, solvate, polymorph, or metabolite thereof is conjugated to the antibody or fragment thereof, as disclosed herein, to form an antibody-drug conjugate (ADC). For example, the filgotinib or a pharmaceutically acceptable salt, solvate, polymorph, or metabolite thereof can be attached by alkylation (e.g., at the epsilon-amino group lysines or the N-terminus of antibodies), reductive amination of oxidized carbohydrate, transesterification between hydroxyl and carboxyl groups, amidation at amino groups or carboxyl groups, and conjugation to thiols. In some embodiments, the number of drug moieties, p, conjugated per antibody molecule ranges from an average of 1 to 8; 1 to 7, 1 to 6, 1 to 5, 1 to 4, 1 to 3, or 1 to 2. In some embodiments, p ranges from an average of 2 to 8, 2 to 7, 2 to 6, 2 to 5, 2 to 4 or 2 to 3. In other embodiments, p is an average of 1, 2, 3, 4, 5, 6, 7 or 8. In some embodiments, p ranges from an average of about 1 to about 8; about 1 to about 7, about 1 to about 6, about 1 to about 5, about 1 to about 4, about 1 to about 3, or about 1 to about 2. In some embodiments, p ranges from about 2 to about 8, about 2 to about 7, about 2 to about 6, about 2 to about 5, about 2 to about 4 or about 2 to about 3.

Filgotinib or a pharmaceutically acceptable salt, solvate, polymorph, or metabolite thereof can be linked to an antibody by a linker. The linker may be a cleavable linker which releases the filgotinib or a pharmaceutically acceptable salt, solvate, polymorph, or metabolite thereof into the cytoplasma once the conjugate is endocytosed by a cell and cleaved, or non-cleavable. A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit), a phenylalanine-lysine (phe-lys) linker, or maleimidocapronic-valine-citruline-p-aminobenzyloxycarbonyl (mc-Val-Cit-PABA) linker. Another linker is Sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its Sulfo-NHS ester is reactive toward primary amines (as found in Lysine and the protein or peptide N-terminus). Yet another linker is maleimidocaproyl (mc). Other suitable linkers include linkers hydrolyzable at a specific pH or a pH range, such as a hydrazone linker. Additional suitable cleavable linkers include disulfide linkers. The linker may be covalently bound to the antibody to such an extent that the antibody must be degraded intracellularly in order for the drug to be released e.g. the mc linker and the like.

Provided herein, in some embodiments, are pharmaceutical compositions and co-formulations comprising filgotinib or a pharmaceutically acceptable salt, solvate, polymorph, or metabolite thereof, and an antibody or fragment thereof, as disclosed herein, such as a selective anti-MMP9 antibody. In various embodiments, the term “co-formulation” may refer to a composition comprising at least two active ingredients, such as filgotinib or a pharmaceutically acceptable salt, solvate, polymorph, or metabolite thereof, and an antibody or fragment thereof, as disclosed herein. Such pharmaceutical compositions and co-formulations are useful, in some embodiments, for administration to a subject in vivo or ex vivo, and for diagnosing and/or treating a subject with an inflammatory disorder.

The conjugates, pharmaceutical compositions, and/or co-formulations disclosed herein can be formulated to be compatible with a particular route of administration, systemic or local. Thus, the conjugates, pharmaceutical compositions, and/or co-formulations can include carriers, diluents, or excipients suitable for administration by various routes. The term “carrier” or “pharmaceutically acceptable carrier” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E.W. Martin. Examples of carriers may include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, silicone emulsion, gum acacia, sterile water, syrup, alginates, tragacanth, calcium silicate, syrup, and polyvinylpyrrolidone. It should be understood, however, that the carriers selected for the pharmaceutical compositions, and the amounts of such carriers in the composition, may vary depending on the method of formulation (e.g., dry granulation formulation, solid dispersion formulation).

Carriers or pharmaceutically acceptable carriers can contain a compound that stabilizes, increases or delays absorption or clearance, in some embodiments. Such compounds include, for example, carbohydrates, such as glucose, sucrose, or dextrans; low molecular weight proteins; compositions that reduce the clearance or hydrolysis of peptides; or excipients or other stabilizers and/or buffers. Agents that delay absorption include, for example, aluminum monostearate and gelatin. Detergents can also be used to stabilize or to increase or decrease the absorption of the pharmaceutical composition, including liposomal carriers. To protect from digestion the compound can be complexed with a composition to render it resistant to acidic and enzymatic hydrolysis, or the compound can be complexed in an appropriately resistant carrier such as a liposome. Means of protecting compounds from digestion are known in the art (see, e.g., Fix (1996) Pharm Res. 13:1760 1764; Samanen (1996) J. Pharm. Pharmacol. 48:119 135; and U.S. Pat. No. 5,391,377, describing lipid compositions for oral delivery of therapeutic agents).

Carriers or pharmaceutically acceptable carriers can also include wetting agents, emulsifying and suspending agents, preserving agents such as methyl and propylhydroxy-benzoates; sweetening agents; and flavoring agents, in some embodiments.

The term “diluent” generally refers to a substance used to dilute the compound of interest prior to delivery. Diluents can also serve to stabilize compounds. Examples of diluents may include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “disintegrant” generally refers to a substance which, upon addition to a solid preparation, facilitates its break-up or disintegration after administration and permits the release of an active ingredient as efficiently as possible to allow for its rapid dissolution. Examples of disintegrants may include maize starch, sodium starch glycolate, croscarmellose sodium, crospovidone, microcrystalline cellulose, modified corn starch, sodium carboxymethyl starch, povidone, pregelatinized starch, and alginic acid.

The term “precipitation inhibitors” generally refers to a substance that prevents or inhibits precipitation of the active agent from a supersaturated solution. One example of a precipitation inhibitor includes hydroxypropylmethylcellulose (HPMC).

The term “surfactants” generally refers to a substance that lowers the surface tension between a liquid and a solid that could improve the wetting of the active agent or improve the solubility of the active agent. Examples of surfactants may include poloxamer and sodium lauryl sulfate.

The term “glidant” generally refers to substances used in tablet and capsule formulations to improve flow-properties during tablet compression and to produce an anti-caking effect. Examples of glidants may include colloidal silicon dioxide, talc, fumed silica, starch, starch derivatives, and bentonite.

The term “binder” generally refers to any pharmaceutically acceptable film which can be used to bind together the active and inert components of the carrier together to maintain cohesive and discrete portions. Examples of binders may include hydroxypropylcellulose, hydroxypropylmethylcellulose, povidone, copovidone, and ethyl cellulose.

The term “lubricant” generally refers to a substance that is added to a powder blend to prevent the compacted powder mass from sticking to the equipment during the tableting or encapsulation process. A lubricant can aid the ejection of the tablet form the dies, and can improve powder flow. Examples of lubricants may include magnesium stearate, stearic acid, silica, fats, calcium stearate, polyethylene glycol, sodium stearyl fumarate, or talc; and solubilizers such as fatty acids including lauric acid, oleic acid, and C8/C10 fatty acid.

The conjugates, pharmaceutical compositions, and/or co-formulations disclosed herein can include pharmaceutically acceptable additives in some embodiments. Examples of additives include, but are not limited to, a sugar such as mannitol, sorbitol, glucose, xylitol, trehalose, sorbose, sucrose, galactose, dextran, dextrose, fructose, lactose and mixtures thereof. Pharmaceutically acceptable additives can be combined with pharmaceutically acceptable carriers and/or excipients such as dextrose. Additives also include surfactants such as polysorbate 20 or polysorbate 80.

The formulation and delivery methods of the conjugates, pharmaceutical compositions, and/or co-formulations disclosed herein may generally be adapted according to the site and the disease to be treated. Exemplary formulations include, but are not limited to, those suitable for parenteral administration, e.g., intravenous, intra-arterial, intramuscular, or subcutaneous administration, or oral or nasal administration.

Conjugates, pharmaceutical compositions, and/or co-formulations, as disclosed herein, for parenteral delivery include, for example, water, saline, phosphate buffered saline, Hank's solution, Ringer's solution, dextrose/saline, and glucose solutions. The conjugates, pharmaceutical compositions, and/or co-formulations can contain auxiliary substances to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. Additional parenteral formulations and methods are described in Bai (1997) J. Neuroimmunol. 80:65 75; Warren (1997) J. Neurol. Sci. 152:31 38; and Tonegawa (1997) J. Exp. Med. 186:507 515. The parenteral preparation can be enclosed in ampules, disposable syringes or multiple dose vials made of glass or plastic.

Conjugates, pharmaceutical compositions, and/or co-formulations, as disclosed herein, for intradermal or subcutaneous administration can include a sterile diluent, such as water, saline solution, fixed oils, polyethylene glycols, glycerine, propylene glycol or other synthetic solvents; antibacterial agents such as benzyl alcohol or methyl parabens; antioxidants such as ascorbic acid, glutathione or sodium bisulfite; chelating agents such as ethylenediaminetetraacetic acid; buffers such as acetates, citrates or phosphates and agents for the adjustment of tonicity such as sodium chloride or dextrose.

Conjugates, pharmaceutical compositions, and/or co-formulations, as disclosed herein, for injection include aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as manitol, sorbitol, and sodium chloride may be included in the composition. The resulting solutions can be packaged for use as is, or lyophilized; the lyophilized preparation can later be combined with a sterile solution prior to administration.

In some embodiments, the conjugates, compositions, and/or co-formulations disclosed herein may be administered orally. Oral administration may be via, for example, capsule or enteric coated tablets. In making the conjugates, pharmaceutical compositions, and/or co-formulations described herein, the active ingredient(s) is(are) usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the conjugates, compositions, and/or co-formulations disclosed herein can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

In the preparation of solid conjugates, pharmaceutical compositions, and co-formulations such as tablets, the principal active ingredient(s) may be mixed with a pharmaceutical excipient to form a solid preformulation composition containing a homogeneous mixture, e.g., of filgotinib or a pharmaceutically acceptable salt, solvate, polymorph, or metabolite thereof. When referring to these preformulation compositions as homogeneous, the active ingredient(s) may be dispersed evenly throughout the composition so that the composition may be readily subdivided into equally effective unit dosage forms such as tablets, pills and capsules.

The tablets or pills comprising at least one of the compounds described herein may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, the conjugates, pharmaceutical compositions, and co-formulations disclosed herein can be formulated so as to provide quick, sustained or delayed release of the active ingredient(s) after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. Such patches may be constructed for continuous, pulsatile, or on demand delivery of pharmaceutical agents.

In some embodiments, the conjugates, pharmaceutical compositions, and/or co-formulations disclosed herein can be combined with other therapeutic moieties or imaging/diagnostic moieties as provided herein. Therapeutic moieties and/or imaging moieties can be provided as a separate composition, or as a conjugated moiety present on an MMP9 binding protein, in certain embodiments.

Conjugates, pharmaceutical compositions, and/or co-formulations, as disclosed herein, for in vivo administration are generally sterile. In one embodiment, the pharmaceutical conjugates, compositions, and/or co-formulations disclosed herein are formulated to be free of pyrogens such that they are acceptable for administration to human patients.

Various other conjugates, pharmaceutical compositions, co-formulations, and techniques for their preparation and use will be known to those of skill in the art in light of the present disclosure. For a detailed listing of suitable pharmacological compositions, co-formulations, and associated administrative techniques one can refer to the detailed teachings herein, which can be further supplemented by texts such as Remington: The Science and Practice of Pharmacy 20th Ed. (Lippincott, Williams & Wilkins 2003).

In some embodiments, the present disclosure provides a pharmaceutical composition comprising: (i) filgotinib or a pharmaceutically acceptable salt, solvate, polymorph, or metabolite thereof; (ii) an antibody or fragment thereof, as disclosed herein, such as a selective anti-MMP9 antibody; and (iii) a pharmaceutically acceptable carrier. In various embodiments, such a pharmaceutical composition exhibits synergy in treating an inflammatory disorder.

In some embodiments, the present disclosure provides a co-formulation comprising: (i) filgotinib or a pharmaceutically acceptable salt, solvate, polymorph, or metabolite thereof; (ii) an antibody or fragment thereof, as disclosed herein, such as a selective anti-MMP9 antibody; and (iii) a pharmaceutically acceptable carrier. In various embodiments, such a co-formulation exhibits synergy in treating an inflammatory disorder.

In some embodiments, the present disclosure provides a composition for use in the treatment of an inflammatory disorder, the composition comprising: (i) filgotinib or a pharmaceutically acceptable salt, solvate, polymorph, or metabolite thereof; and (ii) an antibody or fragment thereof, as described herein, such as a selective anti-MMP9 antibody.

In additional embodiments, the present disclosure provides combination therapy for treating an inflammatory disorder, wherein separate compositions of filgotinib or a pharmaceutically acceptable salt, solvate, polymorph, or metabolite thereof, and an antibody or fragment thereof, as disclosed herein, are used. For example, a composition comprising filgotinib or a pharmaceutically acceptable salt, solvate, polymorph, or metabolite thereof, and a composition comprising an antibody or fragment thereof, as disclosed herein (such as a selective anti-MMP9 antibody), may be used separately for the combination therapy.

As indicated above, the conjugates, pharmaceutical compositions, and/or co-formulations disclosed herein can be suitable for administration locally or systemically by any suitable route (e.g., rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, an artery-inserted cylindrical polymer, etc.).

The conjugates, pharmaceutical compositions, and/or co-formulations disclosed herein can be formulated based on the physical characteristics of the patient/subject needing treatment, the route of administration, and the like. Such can be packaged in a suitable pharmaceutical package with appropriate labels for the distribution to hospitals and clinics wherein the label is for the indication of treating a disorder as described herein in a subject. Medicaments can be packaged as a single or multiple units. Instructions for the dosage and administration of the pharmaceutical compositions of the present invention can be included with the pharmaceutical packages and kits described below.

### Kits

Compositions (including, for example, conjugates, formulations, co-formulations and unit dosages) comprising filgotinib or a pharmaceutically acceptable salt, solvate, polymorph, or metabolite thereof, and compositions comprising an antibody or fragment thereof, as disclosed herein, can be prepared and placed in an appropriate container, and labeled for treatment of an indicated condition. Accordingly, provided herein is also an article of manufacture, such as a container comprising a unit dosage form of filgotinib or a pharmaceutically acceptable salt, solvate, polymorph, or metabolite thereof, and a unit dosage form of an antibody or fragment thereof, as disclosed herein, and a label containing instructions for use of the compounds.

In some embodiments, the article of manufacture is a container comprising: (i) a unit dosage form of filgotinib or a pharmaceutically acceptable salt, solvate, polymorph, or metabolite thereof, and one or more pharmaceutically acceptable carriers, adjuvants or excipients; and (ii) a unit dosage form of an antibody or fragment thereof, as disclosed herein, and one or more pharmaceutically acceptable carriers, adjuvants or excipients.

In some embodiments, the article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of a medical condition. It should be understood that the active ingredient may be packaged in any material capable of improving chemical and physical stability, such as an aluminum foil bag. In some embodiments, diseases or medical conditions indicated on the label can include, for example, treatment of an inflammatory disorder.

Kits also are contemplated. For example, a kit can comprise unit dosage forms of filgotinib or a pharmaceutically acceptable salt, solvate, polymorph, or metabolite thereof, and compositions comprising an antibody or fragment thereof, as described herein, and a package insert containing instructions for use of the composition in treatment of a medical condition, such as an inflammatory disorder.

In some embodiments, the kits comprise (i) a unit dosage form of filgotinib or a pharmaceutically acceptable salt, solvate, polymorph, or metabolite thereof, and one or more pharmaceutically acceptable carriers, adjuvants or excipients; and (ii) a unit dosage form of a an antibody or a fragment thereof, as disclosed herein, and one or more pharmaceutically acceptable carriers, adjuvants or excipients. In certain embodiments, the kit may additionally include instructions for use of (i) and (ii) in treating an inflammatory disorder.

### Dosing

The dosing regimen of filgotinib or a pharmaceutically acceptable salt, solvate, polymorph, or metabolite thereof, and an antibody or fragment thereof, as disclosed herein, in the provided methods may vary depending upon the indication, route of administration, and severity of the condition. For instance, depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the subject, the age, condition, body weight, sex, and diet of the subject, and the severity of any infection. Additional factors that can be taken into account include MMP9 activity, time and frequency of administration, duration of treatment, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

As indicated above, the dose and frequency of dosing may depend on pharmacokinetic and pharmacodynamic, as well as toxicity and therapeutic efficiency data. For example, pharmacokinetic and pharmacodynamic information about filgotinib or a pharmaceutically acceptable salt, solvate, polymorph, or metabolite thereof can be collected through preclinical in vitro and in vivo studies, later confirmed in humans during the course of clinical trials. Thus, for filgotinib or a pharmaceutically acceptable salt, solvate, polymorph, or metabolite thereof, a therapeutically effective dose can be estimated initially from biochemical and/or cell-based assays. The dosage can then be formulated in animal models to achieve a desirable circulating concentration range that modulates JAK expression or activity. As human studies are conducted further information will emerge regarding the appropriate dosage levels and duration of treatment for various diseases and conditions.

For the antibodies or fragments disclosed herein, the dosage, in some embodiments, may be determined based on a pharmacokinetic model for antibodies displaying target-mediated disposition. In contrast to the relatively linear pharmacokinetics observed for antibodies directed to soluble receptor targets, antibodies directed toward tissue-based target receptors frequently demonstrate non-linear pharmacokinetics. Mager, D. E. (2006), Adv Drug Deliv Rev 58(12-13): 1326-1356. The basis for non-linear disposition relates to the high affinity binding of antibody to target and the extent of binding (relative to dose), such that the interaction is reflected in the pharmacokinetic characteristics of the antibody. Mager, D. E. and W. J. Jusko (2001), J Pharmacokinet Pharmacodyn 28(6): 507-532. Included within target mediated drug disposition is receptor-mediated endocytosis (internalization) of the antibody-receptor complex. Wang, W., E. Q. Wang, et al. (2008), Clin Pharmacol Ther 84(5): 548-558.

In a pharmacokinetic model for an antibody having target-mediated disposition, in the absence of drug (antibody), the target receptor is synthesized at a constant rate and eliminated by a first-order process. As a result, the target receptor exists at a steady-state concentration in the absence of drug (antibody). When a drug is added to the body it can interact with the target receptor in a bimolecular reaction, distribute into less well perfused tissue, or be eliminated via first-order processes. At low drug concentrations the predominant movement of the drug is onto the receptor due to the high affinity binding. As the amount of the drug entering the body becomes sufficient to bind the available mass of receptor the drug distributes into and out of tissue and is eliminated. As drug concentrations fall and drug equilibrates from tissue this provides an additional reservoir to binding newly synthesized receptor.

Toxicity and therapeutic efficacy of filgotinib or a pharmaceutically acceptable salt, solvate, polymorph, or metabolite thereof, and/or an antibody or fragment thereof, as disclosed herein, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

Accordingly, the formulation, route of administration, dosage and dosing frequency of filgotinib or a pharmaceutically acceptable salt, solvate, polymorph, or metabolite thereof, and/or an antibody or fragment thereof, as disclosed herein may be based on one or more factors disclosed herein, and tailored to the individual subject, the nature of the condition to be treated in the subject, and generally, the judgment of the attending practitioner. The physician can also start doses of any of the compounds disclosed herein employed in the pharmaceutical compositions and/or co-formulations at levels lower than that required in order to achieve the desired therapeutic effect and gradually increase the dosage until the desired effect is achieved.

In some embodiments, a therapeutically effective amount or a pharmaceutically effective amount refers to an amount that is sufficient to effect treatment, when administered to a subject (e.g., a human) in need of such treatment. In one embodiment, a therapeutically effective amount of filgotinib, or a pharmaceutically acceptable salt, solvate, polymorph, or metabolite thereof, is an amount sufficient to modulate JAK expression, and thereby treat a human suffering an indication, or to ameliorate or alleviate the existing symptoms of the indication. In another embodiment, a therapeutically effective amount of an antibody or fragment, as disclosed herein, is an amount sufficient to modulate expression of MMP9.

In some embodiments, the therapeutically effective amount of any of the compounds disclosed herein may be determined based on data obtained from assays known in the art, including for example, an apoptosis assay.

The therapeutically effective amount of any of the compounds disclosed herein may be provided in a single dose or multiple doses to achieve the desired treatment endpoint. As used herein, “dose” refers to the total amount of an active ingredient (e.g., filgotinib or a pharmaceutically acceptable salt, solvate, polymorph, or metabolite thereof; and/or an antibody or fragment as disclosed herein) to be taken each time by a subject (e.g., a human).

In some embodiments, the compounds disclosed herein may be provided in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. The compounds are generally administered in a pharmaceutically effective amount. For instance, in some embodiments, each dosage unit, for oral administration, contains from about 10 mg to about 1000 mg of a compound disclosed herein, for example from about 50 mg to about 500 mg, for example about 50 mg, about 75 mg, about 100 mg, about 150 mg, about 200 mg, about 250 mg, or about 300 mg. In other embodiments, for parenteral administration, each dosage unit contains from 0.1 to 700 mg of a compound disclosed herein.

The dose of any of the compounds disclosed herein may be administered once daily (QD), twice daily (BID), three times daily, four times daily, or more than four times daily using any suitable mode described herein (e.g., oral administration). In some embodiments, the dose of any of the compounds disclosed herein is administered once daily. In some embodiments, the dose of any of the compounds disclosed herein is administered twice daily.

Moreover, administration or treatment with the compounds disclosed herein may be continued for a number of days; for example, treatment may continue for at least 7 days, 14 days, or 28 days, for one cycle of treatment. Treatment cycles are well known, and are frequently alternated with resting periods of about 1 to 28 days, commonly about 7 days or about 14 days, between cycles. The treatment cycles, in other embodiments, may also be continuous.

In some embodiments, filgotinib, or a pharmaceutically acceptable salt, solvate, polymorph, or metabolite thereof, is administered to a human at a dose between 40 mg and 1200 mg, between 40 mg and 800 mg, between 40 mg and 600 mg, or between 40 mg and 400 mg.

In some embodiments, the therapeutically effective amount of filgotinib, or a pharmaceutically acceptable salt, solvate, polymorph, or metabolite thereof, is administered to a human at a dose of from about 1 mg to about 200 mg, about 10 mg to about 200 mg, about 100 mg to about 200 mg, about 50 mg to about 175 mg, about 20 mg to about 160 mg, about 20 mg to about 150 mg, about 10 mg to about 100 mg, or about 75 mg to about 100 mg. In some embodiments, filgotinib, or a pharmaceutically acceptable salt, solvate, polymorph, or metabolite thereof, is administered to a human at a dose between about 50 mg to about 200 mg.

In some embodiments, filgotinib, or a pharmaceutically acceptable salt, solvate, polymorph, or metabolite thereof, is administered to a human in need thereof in an individual dose of 1 mg, 5 mg, 10 mg, 20 mg, 30 mg, 40 mg, 50 mg, 60 mg, 70 mg, 75 mg, 80 mg, 900 mg, 100 mg, 110 mg, 120 mg, 130 mg, 140 mg, 150 mg, 160 mg, 170 mg, 175 mg, or 200 mg. In additional embodiments, filgotinib, or a pharmaceutically acceptable salt, solvate, polymorph, or metabolite thereof, is administered to a human at an individual dose of about 300 mg, about 400 mg, about 500 mg, about 600 mg, about 700 mg, or about 800 mg. In some embodiments, filgotinib, or a pharmaceutically acceptable salt, solvate, polymorph, or metabolite thereof, is administered to a human in need thereof at an individual dose of about 100 mg. In some embodiments, filgotinib, or a pharmaceutically acceptable salt, solvate, polymorph, or metabolite thereof, is administered to a human in need thereof at an individual dose of about 200 mg.

The doses of filgotinib, or a pharmaceutically acceptable salt, solvate, polymorph, or metabolite thereof, disclosed herein may be administered once daily, twice daily, three times daily, or four or more times daily. For example, in some embodiments, the dosage of filgotinib, or a pharmaceutically acceptable salt, solvate, polymorph, or metabolite thereof, is about 50 mg to about 200 mg once, twice, three times, four times, or more than four times daily. In some embodiments, the dosage of filgotinib, or a pharmaceutically acceptable salt, solvate, polymorph, or metabolite thereof, is about 50 mg to about 200 mg once daily. In some embodiments the dosage of filgotinib, or a pharmaceutically acceptable salt, solvate, polymorph, or metabolite thereof, is about 50 mg, about 75 mg, about 100 mg, about 125 mg, about 175 mg, or about 200 mg once daily.

In certain embodiments, filgotinib, or a pharmaceutically acceptable salt, solvate, polymorph, or metabolite thereof, is formulated as a capsule or a tablet. In one embodiment, the capsule or tablet comprises from about 50 mg to about 500 mg of filgotinib, or a pharmaceutically acceptable salt, solvate, polymorph, or metabolite thereof. In another embodiment, the capsule or tablet comprises from about 50 mg to about 200 mg of filgotinib, or a pharmaceutically acceptable salt, solvate, polymorph, or metabolite thereof. In some embodiments, the capsule or tablet comprises about 10 mg, about 25 mg, about 50 mg, about 75 mg, about 100 mg, about 150 mg, about 200 mg, about 250 mg, about 300 mg, about 350 mg, about 400 mg, about 450 mg, or about 500 mg of filgotinib, or a pharmaceutically acceptable salt, solvate, polymorph, or metabolite thereof.

In some embodiments, the therapeutically effective amount of filgotinib, or a pharmaceutically acceptable salt, solvate, polymorph, or metabolite thereof, may be an amount sufficient to decrease a symptom of a disease or condition responsive to inhibition of JAK activity. For instance, in certain embodiments, filgotinib, or a pharmaceutically acceptable salt, solvate, polymorph, or metabolite thereof, is administered to a human at a dose resulting in about 50%, about 55%, about 60%, about 65%, about 70%, about 75%, about 80%, about 90%, about 95%, or about 99% JAK target inhibition.

In some embodiments, the antibody or fragment thereof, as disclosed herein, may be administered in vivo at a dosage ranging from about 10 ng/kg to up to 100 mg/kg of mammal body weight or more per day, about 1 μg/kg/day to 50 mg/kg/day, about 100 μg/kg/day to 20 mg/kg/day, 500 μg/kg/day to 10 mg/kg/day, or 1 mg/kg/day to 10 mg/kg/day, depending upon the route of administration.

In some embodiments, the antibody or fragment thereof, as disclosed herein, is administered intravenously, for example, at a dose from about 1 mg/kg to about 30 mg/kg. In one embodiment, the antibody or fragment is administered intravenously, for example, at a dose of about 2 mg/kg to about 28 mg/kg. In another embodiment, the antibody or fragment, as disclosed herein, thereof is administered intravenously, for example, at a dose of about 4 mg/kg to about 28 mg/kg. In yet another embodiment, the antibody or fragment thereof, as disclosed herein, is administered intravenously at a dose of about 1 mg/kg to about 14 mg/kg, or about 2 mg/kg to at or about 14 mg/kg, once every 14 days. In some embodiments, the effective amount of dosage of the antibody or fragment thereof, as disclosed herein, is administered once every 7 to 28 days. In one embodiment, the effective amount of dosage of the antibody or fragment thereof, as disclosed herein, is administered once every 7 days. In another embodiment, the effective amount of dosage of the antibody or fragment thereof, as disclosed herein, is administered once every 28 days.

In some embodiments, the antibody or fragment thereof, as disclosed herein, is administered subcutaneously, for example, at a dose from about 1 mg/kg to about 30 mg/kg. In one embodiment, subcutaneous dosages of the antibody or fragment thereof, as disclosed herein, range from at or about 1 mg/kg to about 28 mg/kg, such as from at or about 2 mg/kg to at or about 28 mg/kg, once every 14 days. In another embodiment, the antibody or fragment thereof, as disclosed herein, is administered subcutaneously at a dose of about 1 mg/kg to about 14 mg/kg, such as from at or about 2 mg/kg to at or about 14 mg/kg, once every 14 days. In some embodiments, the effective amount of dosage of the antibody or fragment thereof, as disclosed herein, is administered once every 7 to 28 days. In one embodiment, the effective amount of dosage of the antibody or fragment thereof, as disclosed herein, is administered once every 7 days. In another embodiment, the effective amount of dosage of the antibody or fragment thereof, as disclosed herein, is administered once every 28 days.

In some embodiments, the antibody of fragment thereof, as disclosed herein, is administered at a dose of 100, 200, 400, 600, 1200, or 1800 mg/kg body weight, and in some examples at a dosage of 133, 267, 400, 600 or 1200 mg/kg. In some examples, the antibody or fragment thereof, as disclosed herein, is administered the interval of one, two or three weeks, or once every one, two, or three weeks. In some examples, the appropriate dosage is made with 0.9% sodium chloride.

In some embodiments, a human patient is administered an antibody or fragment thereof, as disclosed herein, intravenously at a dosage of 100, 200, 400, 600, 1200, or 1800 mg/kg body weight, at the interval of one, two or three weeks. In some aspects, the appropriate dosage is made with 0.9% sodium chloride.

In some embodiments, the dosage of an antibody or fragment thereof, as disclosed herein, can be adjusted and administered at 133, 267, 400, 600 or 1200 mg/kg body weight. After each therapeutic cycle, the patients may be monitored for the levels of MMP9 antibodies, MMP9, or other suitable biomarkers.

### EXAMPLE

The following study is conducted to evaluate the efficacy of filgotinib in combination with an MMP9 binding protein in a recognized model of arthritis, the rat type II collagen-induced arthritis model. Lewis rats are injected intradermally/subcutaneously (ID/SC) with porcine type II collagen to induce arthritis. Arthritic rats are treated with vehicle, filgotinib, an anti-MMP9 antibody, or a combination of filgotnib and an anti-MMP9 antibody. Efficacy evaluation is based on body weights, daily ankle caliper measurements, ankle diameter expressed as area under the curve (AUC), terminal hind paw weights, and histopathologic evaluation of right ankles.

This model reflects certain clinical and histopathologic parameters, such as inflammation, cartilage destruction, and bone resorption that occur in established type II collage arthritis in female Lewis rats. As the treatment is initiated at the peak of established disease and continues into the chronic phase, the results obtained may be used in evaluating chronic, highly destructive macrophage-mediated phase of this model.

The combination of filgotinib and an anti-MMP9 antibody is efficacious in this established model of rheumatoid arthritis.

1. A method of treating an inflammatory disorder in a human in need thereof, comprising administering to the human:
(i) a therapeutically effective amount of filgotinib or a pharmaceutically acceptable salt, solvate, polymorph, or metabolite thereof; and
(ii) a therapeutically effective amount of an matrix metalloproteinase 9 (MMP9) binding protein.

2. The method of claim 1, wherein the MMP9 binding protein is a selective anti-MMP9 antibody.

3. The method of claim 2, wherein the antibody is a human antibody or a humanized antibody.

4. The method of claim 2, wherein the antibody comprises a heavy chain variable (VH) region comprising a heavy chain complementary determining region (CDR) with an amino acid sequence selected from the group consisting of SEQ ID NO: 13, SEQ ID NO: 14, SEQ ID NO: 15, and combinations thereof.

5. The method of claim 4, wherein the VH region comprises a heavy chain CDR1 with the amino acid sequence of SEQ ID NO: 13, a heavy chain CDR2 with the amino acid sequence of SEQ ID NO: 14, and a heavy chain CDR3 with the amino acid sequence of SEQ ID NO: 15.

6. The method of claim 2, wherein the antibody comprises a light chain variable (VL) region comprising a light chain complementary determining region (CDR) with an amino acid sequence selected from the group consisting of SEQ ID NO: 16, SEQ ID NO: 17, SEQ ID NO: 18, and combinations thereof.

7. The method of claim 6, wherein the VL region comprises a light chain CDR1 with the amino acid sequence of SEQ ID NO: 16, a light chain CDR2 with the amino acid sequence of SEQ ID NO: 17, and a light chain CDR3 with the amino acid sequence of SEQ ID NO: 18.

8. The method of claim 4, wherein the VH region comprises the amino acid sequence set forth in SEQ ID NO: 1, SEQ ID NO: 3, SEQ ID NO: 5, SEQ ID NO: 6, SEQ ID NO: 7, or SEQ ID NO: 8.

9. The method of claim 6, wherein the VL region comprises the amino acid sequence set forth in SEQ ID NO: 2, SEQ ID NO: 4, SEQ ID NO: 9, SEQ ID NO: 10, SEQ ID NO: 11, or SEQ ID NO: 12.

10. The method of claim 2, wherein the antibody comprises a heavy chain variable (VH) region having the amino acid sequence set forth in SEQ ID NO: 7 and the antibody comprises a light chain variable (VL) region having the amino acid sequence set forth in SEQ ID NO: 12.

11. The method of claim 2, wherein the antibody specifically binds to an epitope of MMP9, wherein the epitope comprises an amino acid residue within a region of MMP9, the region consisting of residues 104-119, residues 159-166, or residues 191-202 of SEQ ID NO: 27.

12. The method of claim 11, wherein the epitope comprises E111, D113, R162, or 1198 of SEQ ID NO: 27.

13. The method of claim 12, wherein the epitope comprises R162 of SEQ ID NO: 27.

14. The method of claim 2, wherein the antibody inhibits the enzymatic activity of MMP9.

15. The method of claim 14, wherein the inhibition of the enzymatic activity is non-competitive.

16. The method of claim 14, wherein the antibody binds MMP9 outside the catalytic domain.

17. The method of claim 1, wherein the inflammatory disorder is rheumatoid arthritis, Crohn's disease, osteoarthritis, allergic airway disease, multiple sclerosis, ulcerative colitis, sepsis, psoriasis, misregulated TNF expression or graft rejection.

18. The method of claim 1, wherein the inflammatory disorder is Crohn's disease.

19. The method of claim 1, wherein the inflammatory disorder is rheumatoid arthritis.

20. The method of claim 1, wherein the inflammatory disorder is ulcerative colitis.

21-27. (canceled)


SEQUENCE LISTING (50 sequences)

SEQ ID NO: 1 - protein, 470 aa, Mus musculus
  CHAIN (1)...(470): AB0041 heavy chain
MAVLVLFLCLVAFPSCVLSQVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 2 - protein, 234 aa, Mus musculus
  CHAIN (1)...(234): AB0041 light chain
MESQIQVFVFVFLWLSGVDGDIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 115 aa, Mus musculus
  CHAIN (1)...(115): variable region of the IgG2b heavy chain of AB0041
QVQLKESGPGLVAPSQSLSITCTVSGFSLLSYGVHWVRQPPGKGLEWLGVIWTGGTTNYNSALMSRLSISKDDSKSQVFLKMNSLQTDDTAIYYCARYYYGMDYWGQGTSVTVSS

SEQ ID NO: 4 - protein, 107 aa, Mus musculus
  CHAIN (1)...(107): variable region of the kappa light chain of AB0041
DIVMTQSHKFMSTSVGDRVSITCKASQDVRNTVAWYQQKTGQSPKLLIYSSSYRNTGVPDRFTGSGSGTDFTFTISSVQAEDLAVYFCQQHYITPYTFGGGTKLEIK

SEQ ID NO: 5 - protein, 115 aa, Artificial Sequence
  synthetic construct
QVQLQESGPGLVKPSETLSLTCTVSGFSLLSYGVHWVRQPPGKGLEWLGVIWTGGTTNYNSALMSRLTISKDDSKSTVYLKMNSLKTEDTAIYYCARYYYGMDYWGQGTSVTVSS

SEQ ID NO: 6 - protein, 115 aa, Artificial Sequence
  synthetic construct
QVQLQESGPGLVKPSETLSLTCTVSGFSLLSYGVHWVRQPPGKGLEWLGVIWTGGTTNYNSALMSRLTISKDDSKNTVYLKMNSLKTEDTAIYYCARYYYGMDYWGQGTLVTVSS

SEQ ID NO: 7 - protein, 115 aa, Artificial Sequence
  synthetic construct
QVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSS

SEQ ID NO: 8 - protein, 115 aa, Artificial Sequence
  synthetic construct
QVQLQESGPGLVKPSETLSLTCTVSGFSLLSYGVHWVRQPPGKGLEWLGVIWTGGTTNYNSALMSRFTISKDDSKNTLYLKMNSLKTEDTAIYYCARYYYGMDYWGQGTLVTVSS

SEQ ID NO: 9 - protein, 107 aa, Artificial Sequence
  synthetic construct
DIVMTQSPSFLSASVGDRVTITCKASQDVRNTVAWYQQKTGKAPKLLIYSSSYRNTGVPDRFTGSGSGTDFTLTISSLQAEDVAVYFCQQHYITPYTFGGGTKVEIK

SEQ ID NO: 10 - protein, 107 aa, Artificial Sequence
  synthetic construct
DIVMTQSPSSLSASVGDRVTITCKASQDVRNTVAWYQQKPGKAPKLLIYSSSYRNTGVPDRFTGSGSGTDFTLTISSLQAEDVAVYFCQQHYITPYTFGGGTKVEIK

SEQ ID NO: 11 - protein, 107 aa, Artificial Sequence
  synthetic construct
DIQMTQSPSSLSASVGDRVTITCKASQDVRNTVAWYQQKPGKAPKLLIYSSSYRNTGVPDRFSGSGSGTDFTLTISSLQAEDVAVYFCQQHYITPYTFGGGTKVEIK

SEQ ID NO: 12 - protein, 107 aa, Artificial Sequence
  synthetic construct
DIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIK

SEQ ID NO: 13 - protein, 10 aa, Artificial Sequence
  synthetic construct
GFSLLSYGVH

SEQ ID NO: 14 - protein, 16 aa, Artificial Sequence
  synthetic construct
VIWTGGTTNYNSALMS

SEQ ID NO: 15 - protein, 7 aa, Artificial Sequence
  synthetic construct
YYYGMDY

SEQ ID NO: 16 - protein, 11 aa, Artificial Sequence
  synthetic construct
KASQDVRNTVA

SEQ ID NO: 17 - protein, 7 aa, Artificial Sequence
  synthetic construct
SSSYRNT

SEQ ID NO: 18 - protein, 9 aa, Artificial Sequence
  synthetic construct
QQHYITPYT

SEQ ID NO: 19 - DNA, 345 bp, Artificial Sequence
  synthetic construct
caggtgcagctgcaggaatccggccctggcctggtcaagccctccgagacactgtccctgacctgcaccgtgtccggcttctccctgctgtcctacggcgtgcactgggtccgacagcctccagggaagggcctggaatggctgggcgtgatctggaccggcggcaccaccaactacaactccgccctgatgtcccggctgaccatctccaaggacgactccaagtccaccgtgtacctgaagatgaactccctgaaaaccgaggacaccgccatctactactgcgcccggtactactacggcatggactactggggccagggcacctccgtgaccgtgtcctca

SEQ ID NO: 20 - DNA, 345 bp, Artificial Sequence
  synthetic construct
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggctgaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 21 - DNA, 345 bp, Artificial Sequence
  synthetic construct
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccgtgtacctgaagatgaactccctgaaaaccgaggacaccgccatctactactgcgcccggtactactacggcatggactactggggccagggcaccctggtcaccgtgtcctca

SEQ ID NO: 22 - DNA, 345 bp, Artificial Sequence
  synthetic construct
caggtgcagctgcaggaatccggccctggcctggtcaagccctccgagacactgtccctgacctgcaccgtgtccggcttctccctgctgtcctacggcgtgcactgggtccgacagcctccaggcaaaggcctggaatggctgggcgtgatctggaccggcggcaccaccaactacaactccgccctgatgtcccggttcaccatctccaaggacgactccaagaacaccctgtacctgaagatgaactccctgaaaaccgaggacaccgccatctactactgcgcccggtactactacggcatggactactggggccagggcaccctggtcaccgtgtcctca

SEQ ID NO: 23 - DNA, 321 bp, Artificial Sequence
  synthetic construct
gacatcgtgatgacccagtcccccagcttcctgtccgcctccgtgggcgacagagtgaccatcacatgcaaggcctctcaggacgtgcggaacaccgtggcctggtatcagcagaaaaccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 24 - DNA, 321 bp, Artificial Sequence
  synthetic construct
gacatcgtgatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggtttaccggctctggctccggcaccgactttaccctgaccatcagctccctgcaggccgaggacgtggccgtgtacttctgccagcagcactacatcaccccctacaccttcggcggaggcaccaaggtggaaataaaa

SEQ ID NO: 25 - DNA, 321 bp, Artificial Sequence
  synthetic construct
gacatccagatgacccagtccccctccagcctgtccgcctctgtgggcgacagagtgaccatcacatgcaaggcctcccaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtacttctgccagcagcactacatcaccccctacaccttcggcggaggcaccaaggtggaaataaaa

SEQ ID NO: 26 - DNA, 321 bp, Artificial Sequence
  synthetic construct
gacatccagatgacccagtccccctccagcctgtccgcctctgtgggcgacagagtgaccatcacatgcaaggcctctcaggacgtgcggaacaccgtggcctggtatcagcagaagcccggcaaggcccccaagctgctgatctactcctcctcctaccggaacaccggcgtgcccgaccggttctctggctctggaagcggcaccgactttaccctgaccatcagctccctgcaggccgaggacgtggccgtgtactactgccagcagcactacatcaccccctacaccttcggcggaggcaccaaggtggaaataaaa

SEQ ID NO: 27 - protein, 707 aa, Homo sapiens
  misc_feature (1)...(707): matrix metalloproteinase 9 (MMP9)
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 28 - protein, 688 aa, Homo sapiens
  misc_feature (1)...(688): mature full-length matrix metalloproteinase 9 (MMP9)
APRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 29 - protein, 19 aa, Homo sapiens
MSLWQPLVLVLLVLGCCFA

SEQ ID NO: 30 - protein, 470 aa, Mus musculus
  CHAIN (1)...(470): M4 heavy chain (IgG2b)
MAVLVLFLCLVAFPSCVLSQVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 31 - protein, 234 aa, Mus musculus
  CHAIN (1)...(234): M4 light chain (kappa)
MESQIQVFVFVFLWLSGVDGDIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 115 aa, Mus musculus
QVQLKESGPGLVAPSQSLSITCTVSGFSLLSYGVHWVRQPPGKGLEWLGVIWTGGSTNYNSALMSRLSISKDDSKSQVFLKMNSLQTDDTAMYYCARYYYAMDYWGQGTSVTVSS

SEQ ID NO: 33 - protein, 107 aa, Mus musculus
DIVMTQSHKFMFTSVGDRVSITCKASQDVRNTVAWYQQKTGQSPKLLIYSASYRNTGVPDRFTGSISGTDFTFTISSVQAEDLALYYCQQHYSTPYTFGGGTKLEVK

SEQ ID NO: 34 - protein, 10 aa, Mus musculus
GFSLLSYGVH

SEQ ID NO: 35 - protein, 10 aa, Mus musculus
VIWTGGSTNY

SEQ ID NO: 36 - protein, 7 aa, Mus musculus
YYYAMDY

SEQ ID NO: 37 - protein, 11 aa, Mus musculus
KASQDVRNTVA

SEQ ID NO: 38 - protein, 7 aa, Mus musculus
SASYRNT

SEQ ID NO: 39 - protein, 9 aa, Mus musculus
QQHYSTPYT

SEQ ID NO: 40 - protein, 229 aa, Mus musculus
  CHAIN (1)...(229): M12 kappa chain
QVFVYMLLWLSGVDGDIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 41 - protein, 107 aa, Mus musculus
DIVMTQSQKFMSTSVGDRVSVTCKASQNVGTNVAWYQQKPGQSPKALIYSASYRFSGVPDRFTGSGSGTDFTLTISNVQSEDLAEYFCQQYNSYPYTFGGGTKLEIK

SEQ ID NO: 42 - protein, 11 aa, Mus musculus
KASQNVGTNVA

SEQ ID NO: 43 - protein, 7 aa, Mus musculus
SASYRFS

SEQ ID NO: 44 - protein, 9 aa, Mus musculus
QQYNSYPYT

SEQ ID NO: 45 - protein, 233 aa, Mus musculus
  CHAIN (1)...(233): AB0046 kappa light chain
MSSAQFLGLLLLCFQGTRCDIQMTQTTSSLSASLGDRVTISCSASQGISNYLNWYQQKPDGTFKLLIYYTSILHSGVPSRFSGSGSGTDYSLTISNLEPEDIATYYCQQYGWLPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 46 - protein, 460 aa, Mus musculus
  CHAIN (1)...(460): AB0046 IgG1 heavy chain
MGWSSIILFLVATATGVHSQVQLQQPGSVLVRPGASVKLSCTASGYTFTSYWMNWVKQRPGQGLEWIGEIYPISGRTNYNEKFKVKATLTVDTSSSTAYMDLNSLTSEDSAVYYCARSRANWDDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 47 - protein, 117 aa, Mus musculus
QVQLQQPGSVLVRPGASVKLSCTASGYTFTSYWMNWVKQRPGQGLEWIGEIYPISGRTNYNEKFKVKATLTVDTSSSTAYMDLNSLTSEDSAVYYCARSRANWDDYWGQGTTLTVSS

SEQ ID NO: 48 - protein, 107 aa, Mus musculus
DIQMTQTTSSLSASLGDRVTISCSASQGISNYLNWYQQKPDGTFKLLIYYTSILHSGVPSRFSGSGSGTDYSLTISNLEPEDIATYYCQQYGWLPRTFGGGTKLEIK

SEQ ID NO: 49 - protein, 461 aa, Artificial Sequence
  synthetic construct
MGWSLILLFLVAVATRVHSQVQLQESGPGLVKPSETLSLTCTVSGFSLLSYGVHWVRQPPGKGLEWLGVIWTGGTTNYNSALMSRFTISKDDSKNTVYLKMNSLKTEDTAIYYCARYYYGMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 234 aa, Artificial Sequence
  synthetic construct
MRVPAQLLGLLLLWLPGARCDIQMTQSPSSLSASVGDRVTITCKASQDVRNTVAWYQQKPGKAPKLLIYSSSYRNTGVPDRFSGSGSGTDFTLTISSLQAEDVAVYYCQQHYITPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC